(19) United States
(12) Patent Application Publication
(10) Pub. No.: US 2024/0423888 A1
(43) Pub. Date: Dec. 26, 2024

(54) COSMETIC AND/OR DERMATOLOGICAL COMPOSITION COMPRISING AT LEAST ONE MEROCYANINE AND AT LEAST ASCORBIC ACID AND/OR A DERIVATIVE THEREOF

(71) Applicant: L'OREAL, Paris (FR)

(72) Inventors: Emilie WARRICK, Aulnay-Sous-Bois (FR); Julien LABOUREAU, Chevilly la Rue (FR); Yang LIU, Shanghai (CN); Marie-Lise CHIRON, Chevilly la Rue (FR)

(73) Assignee: L'OREAL, Paris (FR)

(21) Appl. No.: 18/706,437

(22) PCT Filed: Dec. 12, 2022
(86) PCT No.: PCT/EP2022/085426

§ 371 Date: May 1, 2024

(30) Foreign Application Priority Data
Dec. 17, 2021 (FR) FR2113930

(51) Int. Cl. A61K 8/41; A61K 8/67; A61Q 1/02; A61Q 17/04; A61Q 19/08

(52) U.S. Cl. CPC A61K 8/411; A61K 8/676; A61Q 1/02; A61Q 17/04; A61Q 19/08

(57) ABSTRACT

The present invention relates to a cosmetic and/or dermatological composition, comprising:
a) at least one merocyanine corresponding to formula (I) below, and also the geometrical isomer forms, notably the E/E or E/Z geometrical isomer forms, thereof:

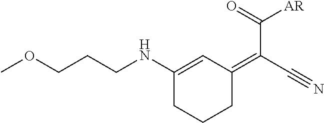

b) ascorbic acid and/or a derivative thereof.

The present invention also relates to a non-therapeutic cosmetic process for caring for and/or making up a keratin material, comprising the application, to the surface of said keratin material, of at least one composition as defined above.

The present invention relates to a cosmetic and/or dermatological composition comprising at least one merocyanine of formula (I), which will be defined in detail below, and ascorbic acid and/or a derivative thereof.

These compositions are preferentially intended for preventing and/or treating the signs of ageing of keratin materials such as the skin, and notably for increasing collagen synthesis.

Human skin consists of two tissues: a surface tissue, the epidermis, and a deep tissue, the dermis.

Natural human epidermis is composed mainly of three types of cells, namely keratinocytes, which form the vast majority, melanocytes and Langerhans cells.

Each of these types of cells contributes, via its intrinsic functions, towards the essential role played in the body by the skin, notably the role of protecting the body against external attacking factors (the climate, ultraviolet rays, smoking, etc.), which is also known as the “barrier function”.

The dermis provides the epidermis with a solid support. It is also its nourishing element. It is constituted mainly of fibroblasts and an extracellular matrix composed predominantly of collagen, elastin and a substance known as ground substance.

These components are synthesized by the fibroblasts. Leukocytes, mast cells and tissue macrophages are also found therein. Finally, blood vessels and nerve fibres pass through the dermis.

The cohesion between the epidermis and the dermis is provided by the dermo-epidermal junction. This is a complex region about 100 nm thick, which comprises the basal pole of the basal keratinocytes, the epidermal membrane and the sub-basal zone of the upper dermis (Bernard P., Structure de la jonction dermo-epidermique. Objectif peau. 2001, 68: 87-93). From a structural viewpoint, hemidesmosomes, in which keratin filaments are inserted (hemidesmosome-tonofilament complex), are distributed on the plasma membrane of the basal keratinocytes. Facing these hemidesmosome-tonofilament complexes are anchoring filaments which cross the epidermal basement membrane. The anchoring filaments are connected to laminin 5 on the epidermal side. Finally, anchoring fibrils constitute the sub-basal network. These are curvilinear structures which originate and terminate on the deep face of the basement membrane and in which collagen I, III and V fibres engage. It has been shown that these anchoring fibrils, which can be perfectly visualized by electron microscopy, are composed of type VII collagen (collagen VII hereinbelow). Collagen VII is synthesized by keratinocytes and fibroblasts, but to a greater extent by keratinocytes (Aumailley M, Rousselle P. Laminins of the dermoepdiermal junction. Matrix Biology. 1999, 18: 19-28; Nievers M, Schaapveld R, Sonnenberg A. Biology and function of hemidesmosomes, Matrix Biology, 1999, 18: 5-17.

Thus, collagens are the major proteins of the extracellular matrices. To date, 20 types of collagen have been identified, and are noted from I to XX. Different collagen families exist within these types, depending on the structures formed:
the family of fibrillar collagens (type I, II, II V/XI) which thus form fibres;
the family of collagens forming the network of the five basal membranes, which comprises type VI and type XVII collagen;
collagens forming hexagonal networks (type VIII and type X), beaded filaments (type VI), FACIT (type IX, XII, XIV, XVI, XIX, XX);
anchoring fibrils which correspond to type VII;
multiplexins (type XV, XVII) and
type XIII collagen, the precise functions of which are not known at present.

In the skin, the collagens predominantly present throughout the dermis are type I and III collagens, which form the extracellular matrix of the entire dermis (these collagens constitute 70-80% of the dry weight of the dermis).

Moreover, collagens are not all synthesized by the same cell types: type I and III collagens are essentially produced by the dermal fibroblasts, whereas type VII collagen is produced by the epidermal keratinocytes. Finally, the regulation of their expression differs from one collagen to another: for example, collagens I and VII are not regulated in the same manner by certain cytokines. Specifically, TNF alpha and leukoregulin stimulate collagen VII and negatively regulate collagen I.

Finally, all collagen molecules are variants of a common precursor which is the a chain of procollagen.

With age, collagen becomes thinner and wrinkles appear on the surface of the skin. Cutaneous ageing is a genetically programmed mechanism. Moreover, certain environmental factors such as smoking and especially exposure to sunlight accelerate it. The skin thus has a much older appearance on the areas exposed to sunlight, such as the back of the hands or the face. Thus, these other factors also have a negative impact on the natural collagen of the skin.

Consequently, in view of the decisive role of collagen, and notably type I collagen, in the cohesion between the epidermal and dermal tissues, and consequently in the integrity of the skin and its resistance to external attacking factors of mechanical type, stimulating the synthesis of these various forms of collagen appears to be an effective means for alleviating the signs of skin ageing.

The aim of the present invention is, precisely, to propose a new cosmetic and/or dermatological composition for limiting the signs of ageing of keratin materials, such as the skin, whether chronobiological or photo-induced, and notably ageing generated by a reduction in skin elasticity and/or by degradation of the collagen in the structure of cutaneous tissues.

However, it is very difficult to find a cosmetic and/or dermatological composition that is especially suitable for preventing and/or treating the signs of ageing of keratin materials such as the skin, for improving the quality of the skin such as its mechanical elasticity properties, while at the same time presenting good cosmetic and sensory properties, notably for example freshness and a glidant sensation on application or a pleasant (notably soft) skin finish.

In the light of the foregoing, it is thus apparent that there remains a consumer need for cosmetic and/or dermatological compositions that are suitable for preventing and/or treating the signs of ageing of keratin materials such as the skin, while at the same time having satisfactory cosmetic properties, in particular with regard to their sensory properties on application, in particular advantageous properties in terms of sensory feel.

The present invention is precisely directed towards meeting these needs.

The Applicant has discovered, surprisingly, that by combining merocyanines of formula (I) defined below with ascorbic acid and/or a derivative thereof, it is possible to substantially improve the action of ascorbic acid and/or a derivative thereof when exposed to UV-A radiation, notably due to the surprising improved effect on the production of procollagen of this combination.

The Applicant has discovered, surprisingly, that a cosmetic and/or dermatological composition comprising at least one merocyanine of formula (I) as defined below and at least ascorbic acid and/or a derivative thereof makes it possible to prevent and/or treat the signs of ageing of a keratin material, such as the skin, in an effective manner, notably when exposed to UV-A radiation, while notably exhibiting good cosmeticity.

In particular, the Applicant has discovered that these cosmetic and/or dermatological compositions according to the invention comprising at least one merocyanine of formula (I) as defined below, and at least ascorbic acid and/or a derivative thereof, and comprising in particular UVA-screening agents in a content of less than 10% by weight relative to the total weight of the composition, more particularly less than 5% by weight, make it possible to prevent and/or treat the signs of ageing of a keratin material, such as the skin, effectively, while exhibiting good cosmetic properties, the latter notably being less greasy and less tacky than equivalent compositions comprising higher contents of organic UVA-screening agents.

The present invention also relates to a non-therapeutic cosmetic process for caring for and/or making up a keratin material, comprising the application, to the surface of said keratin material, of at least one composition according to the invention as defined above.

The invention also relates to a non-therapeutic cosmetic process for preventing and/or treating the signs of ageing, such as the signs of photo-ageing, of a keratin material, comprising the application, to the surface of the keratin material, of at least one composition as defined previously.

The present invention also relates to the use of at least one merocyanine of formula (I) as defined below combined with ascorbic acid and/or a derivative thereof in order to reduce or treat the signs of ageing, notably the cutaneous signs of photo-ageing, in particular to improve the elasticity, firmness and evenness of the complexion, the microrelief and/or wrinkles and fine lines and/or to reduce the signs associated with the elastosis of keratin materials such as the skin.

The combination according to the invention is effective in combating the signs of skin ageing more effectively and/or stimulating skin regeneration/repair processes. The combination according to the invention thus has a particular application in cosmetic compositions intended for preventing and/or cosmetically treating skin ageing, notably for preventing and/or treating, in particular topically, the cutaneous signs of ageing, and most particularly the cutaneous signs associated with wrinkled skin, skin showing a reduction in its viscoelastic or biomechanical properties, and/or skin showing a change in its surface appearance.

Specifically, it has been found, surprisingly, that the merocyanines of formula (I), as defined below, are capable of stimulating and potentiating the action of ascorbic acid and/or a derivative thereof on the production of procollagen I by fibroblasts.

Other characteristics, aspects and advantages of the invention will become apparent on reading the detailed description that follows.

The composition according to the invention is intended for topical application and thus contains a physiologically acceptable medium. The term “physiologically acceptable medium” means here a medium that is compatible with keratin materials. In the context of the present invention, the term “keratin material” notably means the skin, scalp, keratin fibres, such as the eyelashes, eyebrows, head hair and bodily hair, nails, mucous membranes, such as the lips, and more particularly the skin and mucous membranes (body, face, area around the eyes, eyelids, lips, preferably body, face and lips).

According to the invention, the term “preventing” or “prevention” means reducing the risk of occurrence or slowing down the occurrence of a given phenomenon, namely, according to the present invention, the signs of ageing of a keratin material.

The use of a composition according to the invention comprising the merocyanines of formula (I) as defined below and ascorbic acid and/or a derivative thereof, can make it possible more particularly to maintain and/or restore the biomechanical properties of the skin, and/or to prevent and/or treat the signs of ageing of the skin. The term “biomechanical properties of the skin” means herein the stretchability, tonicity, firmness, suppleness and/or elasticity properties of the skin.

The term “signs of ageing of the skin” notably means any modification of the outer appearance of the skin due to ageing, whether it is chronobiological and/or extrinsic ageing, in particular photoinduced or hormonal ageing; among these signs, it is possible to distinguish:
wrinkled skin, which is notably reflected by the appearance of wrinkles and/or fine lines;
skin displaying impairment of its viscoelastic or biomechanical properties, or skin exhibiting a lack of elasticity and/or of stretchability and/or of firmness and/or of suppleness and/or of tonicity, which is notably reflected by wizened, flaccid, slack or saggy skin;
skin displaying impairment of the cohesion of its tissues;
thinned skin; and
skin displaying impairment of its surface appearance, which is notably reflected by impairment of the grain of the skin, for example roughness.

The invention relates to the non-therapeutic use of one or more compounds of formula (I) with ascorbic acid and/or a derivative thereof according to the invention, as an agent for preventing and/or reducing the signs of ageing of the skin.

Thus, according to a first aspect, the present invention relates to a cosmetic and/or dermatological composition, in particular for making up and/or caring for keratin materials, comprising:
at least one merocyanine of formula (I) below and also the E/E- or E/Z-geometrical isomer forms thereof:

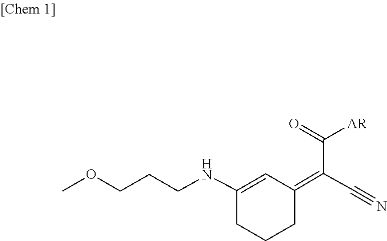

in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, said groups possibly being interrupted with one or more O; and
at least ascorbic acid and/or a derivative thereof.

According to another of its aspects, the present invention also relates to a cosmetic process, in particular a non-therapeutic process, for caring for keratin materials, in particular of the body and/or of the face, comprising at least one step of applying a composition as defined above to said keratin materials.

Other characteristics, aspects and advantages of the invention will become apparent on reading the detailed description that follows.

A composition according to the invention is cosmetic and/or dermatological, and preferably is cosmetic.

A composition according to the invention is generally suitable for topical application to the skin and thus generally comprises a physiologically acceptable medium, i.e. a medium that is compatible with the skin.

It is preferably a cosmetically acceptable medium, i.e. a medium which has a pleasant colour, odour and feel and which does not cause any unacceptable discomfort, i.e. stinging, tautness or redness, liable to discourage the user from applying this composition.

### Merocyanines

As mentioned previously, a composition according to the invention comprises at least one merocyanine of formula (I) below, also the E/E- or E/Z-geometrical isomer forms thereof:

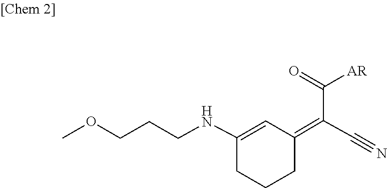

in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, said groups possibly being interrupted with one or more O.

As indicated above, the merocyanine compounds of the invention may be in their E/E- or E/Z-geometrical isomer forms:

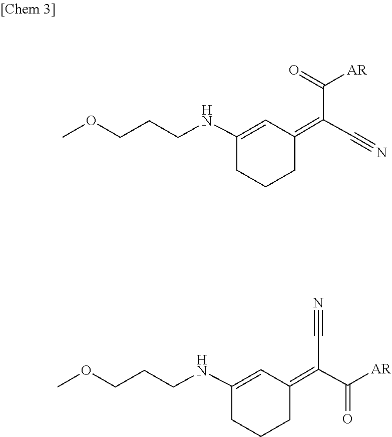

Preferably, in the compounds of formula (I), A is —O— and R is a C₁-C₂₂ alkyl which may be interrupted with one or more O.

Among the compounds of formula (I), use will more particularly be made of at least one compound chosen from the following compounds and also the E/E- or E/Z-geometrical isomer forms thereof:

TABLE 1

| | |
|---|---|
| (A) | 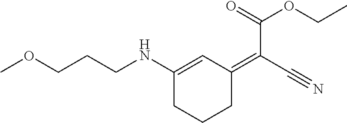 [SMILES: CCOC(=O)/C(C#N)=C1\C=C(NCCCOC)CCC1] |
| | ethyl (2Z)-cyano{3-[(3- |
| | methoxypropyl)amino]cyclohex- |
| | 2-en-1-ylidene}ethanoate |
| (B) | 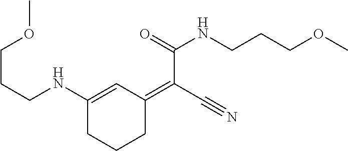 [SMILES: COCCCNC(=O)/C(C#N)=C1\C=C(NCCCOC)CCC1] |
| | (2Z)-2-cyano-N-(3- |
| | methoxypropyl)-2-{3-[(3- |
| | methoxypropyl)amino]cyclohex- |
| | 2-en-1-ylidene}ethanamide |
| (C) | 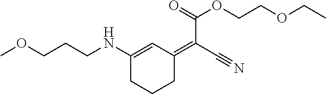 [SMILES: CCOCCOC(=O)/C(C#N)=C1\C=C(NCCCOC)CCC1] |
| | 2-ethoxyethyl (2Z)-cyano{3-[(3- |
| | methoxypropyl)amino]cyclohex- |
| | 2-en-1-ylidene}ethanoate |
| (D) | 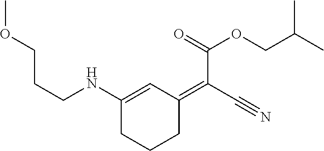 [SMILES: COCCCNC1=C/C(=C(/C#N)C(=O)OCC(C)C)CCC1] |
| | 2-methylpropyl (2Z)-cyano{3-[(3- |
| | methoxypropyl)amino]cyclohex- |
| | 2-en-1-ylidene}ethanoate |
| (E) | 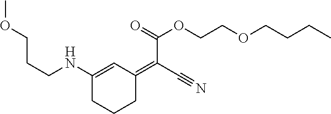 [SMILES: CCCCOCCOC(=O)/C(C#N)=C1\C=C(NCCCOC)CCC1] |
| | 2-butoxyethyl (2Z)-cyano{3-[(3- |
| | methoxypropyl)amino]cyclohex- |
| | 2-en-1-ylidene}ethanoate |
| (F) | 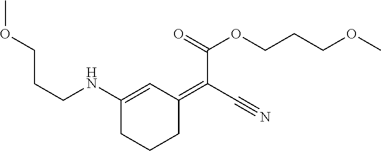 [SMILES: COCCCNC1=C/C(=C(/C#N)C(=O)OCCCOC)CCC1] |
| | 3-methoxypropyl (2Z)-cyano{3- |
| | [(3- |
| | methoxypropyl)amino]cyclohex- |
| | 2-en-1-ylidene}ethanoate |
| (G) | 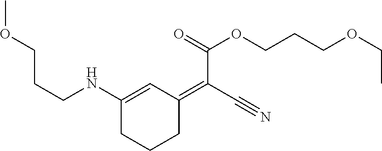 [SMILES: CCOCCCOC(=O)/C(C#N)=C1\C=C(NCCCOC)CCC1] |
| | 3-ethoxypropyl (2Z)-cyano{3-[(3- |
| | methoxypropyl)amino]cyclohex- |
| | 2-en-1-ylidene}ethanoate. |

According to a more particularly preferred mode of the invention, use will be made of compounds (A) and/or (C) and mixtures thereof, and even more preferentially compound (C) in its E/E and/or E/Z geometrical configuration.

Thus, preferably, a composition according to the invention comprises 2-ethoxyethyl (2Z)-cyano{3-[(3-methoxypropyl)amino]cyclohex-2-en-1-ylidene}ethanoate (C), also known as Methoxypropylamino Cyclohexenylidene Ethoxyethylcyanoacetate, in its E/E and/or E/Z geometrical configuration.

The E/Z form has the following structure:

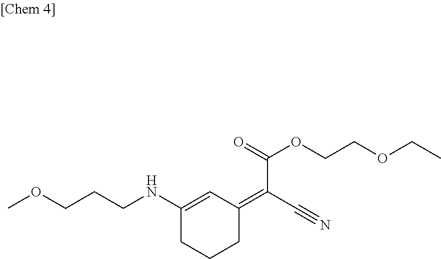 [SMILES: CCOCCOC(=O)/C(C#N)=C1\C=C(NCCCOC)CCC1]

The E/E form has the following structure:

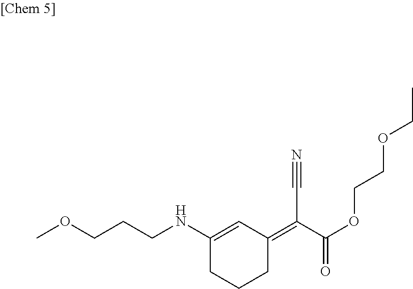 [SMILES: CCOCCOC(=O)/C(C#N)=C1/C=C(NCCCOC)CCC1]

According to a preferred embodiment, a composition according to the invention comprises at least one merocyanine of formula (I) as defined above, in a content ranging from 0.1% to 10% by weight, preferentially from 0.2% to 10% by weight, more preferentially from 0.5% to 10% by weight, relative to the total weight of the composition.

In general, merocyanine-type compounds may be prepared according to known processes, as described, for example, in J. Org. Chem. USSR (English Translation) 26(8), p. 1562f (1990); J. Heterocycl. Chem. 33(3), pages 763-766 (1996); Khimiya Geterotsiklicheskikh Soedinenii 11, pages 1537-1543 (1984); Khimiya Geterotsiklicheskikh Soedinenii 3, pages 397-404 (1982); Chem. Heterocycl. Comp. (English Translation), 24(8), 914-919 (1988), and in Synthetic Communications, Vol. 33, No. 3, 2003, pages 367-371.

As regards compounds of formula (I) more particularly considered according to the invention, and which have the specific feature of possessing a carbocyclic ring containing 6 carbon atoms, they may be prepared according to the protocols described in patent application WO 2007/071582, in IP.com Journal (2009), 9(5A), 29-30 IPCOM000182396D under the title “Process for producing 3-amino-2-cyclohexan-1-ylidene compounds” and in U.S. Pat. No. 4,749,643 on col. 13, line 66-col. 14, line 57, and the references cited in this regard.

In particular, compounds of formula (I) in Table 1 above can be synthesized according to the synthetic scheme described in B. Winkler et al., Tetrahedron Letters, 55 (2014) 1749-1751, entitled “A cyclic merocyanine UV-A absorber: mechanism of formation and crystal structure”, and shown below, for compounds of formula (I):

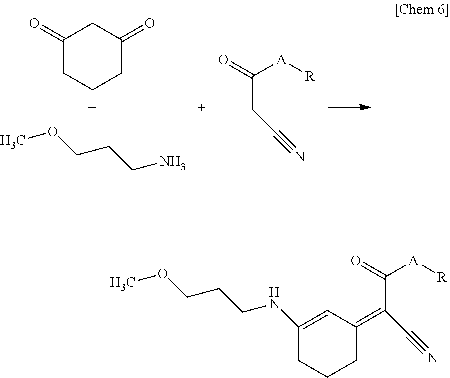

As regards compound (C) of Table 1, the following synthetic scheme is preferred.

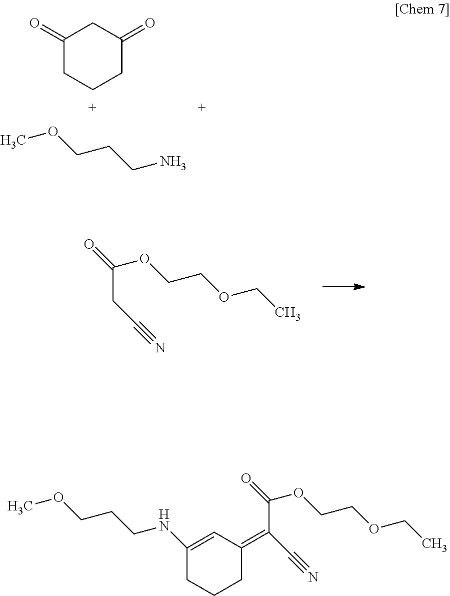

The synthesis illustrated by this scheme is notably detailed in example A4 below.

Ascorbic Acid and/or Derivatives

As mentioned previously, a composition according to the invention comprises ascorbic acid, also known as vitamin C, and/or of a derivative thereof.

In particular, a composition according to the invention comprises from 0.01% to 30% by weight, more particularly from 2% to 20% by weight, and preferably from 5% to 15% by weight, of ascorbic acid, also known as vitamin C, and/or a derivative thereof, and more preferentially of ascorbic acid, relative to the total weight of the composition.

Preferably, a composition according to the invention comprises from 2% to 20% by weight of active material, and more preferentially from 5% to 15% by weight of active material, of ascorbic acid, also known as vitamin C, and/or of a derivative thereof, relative to the total weight of the composition.

More preferentially, a composition according to the invention comprises from 2% to 20% by weight of active material, and more preferentially from 5% to 15% by weight of active material, of ascorbic acid, relative to the total weight of the composition. Ascorbic acid may be in D or L form, advantageously in L form.

By way of example, an ascorbic acid according to the invention is sold by the company Northeast General Pharmaceutical Factory under the trade name Ascorbic Acid EP/BP/USP/FCC/E300®, by the company CSPC Weisheng Pharmaceutical under the name AscorbicAcid 100 MESH® or by the company DSM under the name Ascorbic Acid Ultra Fine Powder®.

The ascorbic acid derivatives according to the invention may be chosen from salts, esters, ethers and sugars thereof.

The salts of ascorbic acid may notably be chosen from sodium ascorbate, magnesium or sodium ascorbyl phosphate, ethers thereof, notably such as acetic, propionic or palmitic esters thereof, or sugars thereof, notably such as glycosylated ascorbic acid.

On account of its chemical structure (alpha-keto lactone), which makes it very sensitive, it may be advantageous to use ascorbic acid in the form of an ascorbic acid sugar ester or a phosphorylated ascorbic acid metal salt.

The sugar esters of ascorbic acid that may be used in the invention are notably glycosyl, mannosyl, fructosyl, fucosyl, galactosyl, N-acetylglucosamine and N-acetylmuramic derivatives of ascorbic acid and mixtures thereof and more especially ascorbyl-2 glucoside or 2-O-α-D-glucopyranosyl L-ascorbic acid or 6-O-β-D-galactopyranosyl L-ascorbic acid. The latter compounds and processes for preparing them are described in particular in EP-A-487 404, EP-A-425 066 and JP05213736.

The phosphorylated ascorbic acid metal salt may be chosen from alkali metal ascorbyl phosphates, alkaline-earth metal ascorbyl phosphates and transition metal, magnesium, sodium, potassium, calcium and zinc ascorbyl phosphates.

According to a particular embodiment, the ascorbic acid derivative is chosen from ascorbyl-2-glucoside, magnesium ascorbyl phosphate and 3-O-ethyl ascorbic acid. More particularly, the ascorbic acid derivative is chosen from ascorbyl-2 glucoside (or 2-O-α-D glucopyranosyl of L-ascorbic acid) and magnesium ascorbyl phosphate. An ascorbic acid derivative may be 3-O-ethyl ascorbic acid (or ethyl ascorbic acid, or L-ascorbic acid, 3-O-ethyl ether).

By way of example, ascorbyl-2 glucoside (or 2-O-α-D-glucopyranosyl L-ascorbic acid) is sold by the company Northeast General Pharmaceutical Factory under the trade name L-Ascorbic Acid 2-Glucoside® (comprising 100% ascorbyl-2 glucoside active material), or by the company Hayashibara under the trade name L-Ascorbic Acid 2-Glucoside® (comprising 100% ascorbyl-2 glucoside active material).

By way of example, 3-O-ethyl ascorbic acid (or ethyl ascorbic acid) is sold by the company Nippon Hypox Laboratories under the trade name Vitamin C Ethyl® (comprising 100% 3-O-ethyl ascorbic acid active material).

By way of example, magnesium ascorbyl phosphate (73% active material in water) is sold by the company Showa Denko under the trade name Ascorbyl PM®, or by the company Jingjiang Hengtong Bio-Engineering under the trade name Magnesium Ascorbyl Phosphate®, or by the company Merck under the trade name Ronacare MAP®.

### Aqueous Phase

A composition according to the invention may comprise an aqueous phase and optionally an oily phase.

The aqueous phase comprises water and optionally a water-soluble organic solvent and is preferably single-phase.

According to a preferred embodiment, the composition according to the present invention has a water content of between 10% and 98% by weight and preferably between 25% and 90% by weight, and more preferentially between 35% and 85%, relative to the total weight of the composition.

According to the present invention, the term “water-soluble organic solvent” denotes a compound that is liquid at room temperature and water-miscible (miscibility with water of greater than 50% by weight at 25° C. and atmospheric pressure).

The water-soluble solvents that may be used in the composition of the invention may also be volatile. In particular, in order to obtain compositions having a BC3 content >0.9% use will be made of a water-soluble cosolvent in addition to the hydrotropes.

Among the water-soluble solvents that can be used in the composition according to the invention, mention may be made most particularly of lower monoalcohols containing from 1 to 5 carbon atoms, such as ethanol and isopropanol, polyols and also alkylene carbonates.

According to a preferred embodiment, the composition according to the invention also comprises at least one alcohol, in particular a monoalcohol, and preferably ethanol.

Preferably, in a composition according to the invention, the alcohol may be present in a content ranging from 0.5% to 30% by weight, better still from 2.0% to 25% by weight, preferably from 3.0% to 15% by weight, relative to the total weight of said composition.

According to an embodiment variant, the aqueous phase of a composition according to the invention may comprise at least one C₂-C₃₂ polyol.

For the purposes of the present invention, the term “polyol” should be understood as meaning any organic molecule including at least two free hydroxyl groups.

Preferably, a polyol in accordance with the present invention is present in liquid form at room temperature.

A polyol that is suitable for use in the invention may be a compound of linear, branched or cyclic, saturated or unsaturated alkyl type, bearing on the alkyl chain at least two —OH functions, in particular at least three —OH functions and more particularly at least four —OH functions.

The polyols that are suitable for formulating a composition according to the present invention are in particular those notably containing from 2 to 32 carbon atoms and preferably 3 to 16 carbon atoms.

The polyol may be chosen, for example, from ethylene glycol, pentaerythritol, trimethylolpropane, propylene glycol, 1,3-propanediol, butylene glycol, isoprene glycol, pentylene glycol, hexylene glycol, C₃ and C₄ ketones and C₂-C₄ aldehydes, caprylyl glycol, glycerol, polyglycerols, such as glycerol oligomers, for instance diglycerol, polyethylene glycols, and mixtures thereof.

According to a preferred embodiment of the invention, said polyol is chosen from ethylene glycol, pentaerythritol, trimethylolpropane, pentylene glycol, propylene glycol, dipropylene glycol (DPG), caprylyl glycol, glycerol, polyglycerols, polyethylene glycols (PEG), and mixtures thereof.

According to a particular embodiment, the composition of the invention may comprise at least one polyol, in particular chosen from glycerol, caprylyl glycol, propylene glycol, pentylene glycol, dipropylene glycol (DPG), PEG-8, and mixtures thereof.

According to a preferred embodiment, the composition of the invention may comprise at least one polyol, in particular chosen from propylene glycol, pentylene glycol, dipropylene glycol (DPG), and mixtures thereof.

Preferably, the composition of the invention may comprise at least dipropylene glycol (DPG), as polyol.

When they are present, the polyol(s) are preferably present in a composition according to the invention in a content ranging from 0.5% to 40% by weight, better still from 10% to 20% by weight, preferably from 5% to 15% by weight and preferentially from 7% to 10% by weight, relative to the total weight of said composition.

According to another particular embodiment, the composition of the invention may comprise at least one alkylene carbonate, notably chosen from those of formula (II) below:

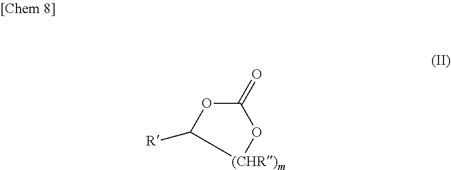

in which:
R′ denotes a hydrogen atom, a linear or branched C₁-C₆ alkyl radical or a linear or branched C₁-C₄ hydroxyalkyl radical;
R″ represents a hydrogen atom, a linear or branched C₁-C₆ alkyl radical or a linear or branched C₁-C₄ hydroxyalkyl radical; and
m is 1, 2 or 3.

Preferably, the radical R′ represents a hydrogen atom, a linear or branched C₁-C₄ alkyl radical or a linear or branched C₁-C₂ hydroxyalkyl radical.

R″ represents a hydrogen atom, a linear or branched C₁-C₂ alkyl radical or a linear or branched C₁-C₂ hydroxyalkyl radical.

Preferably, m is 1.

As particularly advantageous examples of alkylene carbonates, mention may be made of the compounds for which the radical R′ represents a hydrogen atom (corresponding to ethylene carbonate), a methyl group (corresponding to propylene carbonate), an ethyl group (corresponding to 1,2-butylene carbonate), a hydroxymethyl group (R′=—CH₂OH; corresponding to glyceryl carbonate).

Preferably, the alkylene carbonate used is propylene carbonate.

The alkyl or alkylene carbonates may be present in the compositions according to the invention at concentrations ranging preferably from 0.1% to 98% by weight, particularly from 0.5% to 50% by weight, more preferentially from 1% to 20% by weight and even more particularly from 1% to 10% by weight, notably 1% to 6% by weight, relative to the total weight of the composition.

According to another particular embodiment, a composition according to the invention may also comprise at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, ibuprofen sodium, sodium xylene sulfonate (SXS), ethyl carbamate, pyridoxal hydrochloride, sodium benzoate, N,N-dimethylacetamide, N-methylacetamide, isoniazid, and mixtures thereof.

According to a particular embodiment, a composition according to the invention comprises at least one hydrotrope chosen from nicotinamide, caffeine, salicylic acid salts and mixtures thereof.

The salicylic acid salts may notably be chosen from sodium salicylate, lysine salicylate, arginine salicylate, magnesium salicylate, and mixtures thereof.

Preferably, the salicylic acid salt is sodium salicylate.

According to another particular embodiment, a composition according to the invention comprises at least nicotinamide as hydrotrope.

The content of hydrotrope(s) according to the invention present in the compositions according to the invention may range from 0.1% to 20% by weight, in particular from 0.1% to 10% by weight, preferably from 0.5% to 10% by weight, notably from 0.5% to 3% by weight relative to the total weight of the composition.

Thus, according to one variant embodiment, a composition according to the invention may contain, along with at least one hydrotrope, at least one organic solvent notably chosen from alcohols, in particular polyols and alkylene carbonates. Besides the abovementioned solvents, a composition according to the invention may also comprise at least one organic solvent chosen from:
ketones that are liquid at room temperature, such as methyl ethyl ketone, methyl isobutyl ketone, diisobutyl ketone, isophorone, cyclohexanone or acetone;
cyclic ethers, such as y-butyrolactone;
short-chain esters (containing from 3 to 8 carbon atoms in total), such as ethyl acetate, butyl acetate, methyl acetate, propyl acetate, isopropyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides, such as dimethyl sulfoxide;
aldehydes that are liquid at room temperature, such as benzaldehyde or acetaldehyde;
ethyl 3-ethoxypropionate;
acetals, such as methylal; and
mixtures thereof.

The aqueous phase may also comprise any water-soluble or water-dispersible compound that is compatible with an aqueous phase, such as stabilizers, gelling agents, film-forming polymers, thickeners, surfactants, and mixtures thereof.

Preferably, the aqueous phase is present in a composition according to the invention in a content ranging from 1% to 100% by weight, preferably from 20% to 95% by weight, and more preferentially from 30% to 90% by weight, relative to the total weight of said composition.

### Fatty Phase

As mentioned above, a composition according to the invention may comprise at least one fatty phase.

For the purposes of the invention, the term “fatty phase” means a phase comprising at least one oil and all of the liposoluble and lipophilic ingredients and the fatty substances used for the formulation of the compositions of the invention.

In particular, a composition according to the invention may comprise from 5% to 95% by weight and preferably from 10% to 80% by weight of fatty phase, relative to the total weight of the composition.

The fatty phase of the composition according to the invention may comprise oils, waxes, pasty compounds, and/or silicone compounds, and preferably at least one oil, notably a cosmetic oil.

### Oils

The term “oil” means a water-immiscible non-aqueous compound that is liquid at room temperature (20° C.) and at atmospheric pressure (760 mmHg).

A fatty phase that is suitable for preparing the compositions, notably cosmetic compositions, according to the invention may comprise hydrocarbon-based oils, silicone oils, fluoro oils or non-fluoro oils, or mixtures thereof.

The oils may be volatile or non-volatile.

They may be of animal, plant, mineral or synthetic origin.

The fatty phase can comprise, besides the merocyanine screening agent(s) and optionally the lipophilic additional screening agent(s), at least one volatile or non-volatile hydrocarbon-based oil and/or one volatile and/or non-volatile silicone oil and/or one volatile and/or non-volatile fluoro oil.

For the purposes of the present invention, the term “hydrocarbon-based oil” means an oil mainly containing hydrogen and carbon atoms.

The term “silicone oil” means an oil comprising at least one silicon atom and notably at least one Si—O group.

The term “fluoro oil” means an oil comprising at least one fluorine atom.

The oils may optionally comprise oxygen, nitrogen, sulfur and/or phosphorus atoms, for example in the form of hydroxyl or acid radicals.

### Volatile Oils

For the purposes of the invention, the term “volatile oil” means any oil that is capable of evaporating on contact with the skin in less than one hour, at room temperature and atmospheric pressure. The volatile oil is a volatile cosmetic compound, which is liquid at room temperature, notably having a non-zero vapour pressure, at room temperature and atmospheric pressure, notably having a vapour pressure ranging from 0.13 Pa to 40 000 Pa (10⁻³ to 300 mmHg), in particular ranging from 1.3 Pa to 13 000 Pa (0.01 to 100 mmHg) and more particularly ranging from 1.3 Pa to 1300 Pa (0.01 to 10 mmHg).

The volatile oils may be hydrocarbon-based oils or silicone oils.

Among the volatile hydrocarbon-based oils containing from 8 to 16 carbon atoms, mention may be made notably of branched C₈-C₁₆ alkanes, for instance C₈-C₁₆ isoalkanes (also known as isoparaffins), isododecane, isodecane, isohexadecane and, for example, the oils sold under the trade names Isopar or Permethyl, branched C₈-C₁₆ esters, for instance isohexyl neopentanoate, and mixtures thereof.

Mention may also be made of volatile linear alkanes comprising from 8 to 16 carbon atoms, in particular from 10 to 15 carbon atoms, and more particularly from 11 to 13 carbon atoms, for instance n-dodecane (C₁₂) and n-tetradecane (C₁₄) sold by Sasol under the respective references Parafol 12-97 and Parafol 14-97, and also mixtures thereof, the undecane-tridecane mixture, mixtures of n-undecane (C₁₁) and of n-tridecane (C₁₃) obtained in examples 1 and 2 of patent application WO 2008/155059 from the company Cognis, and mixtures thereof.

Volatile silicone oils that may be mentioned include linear volatile silicone oils such as hexamethyldisiloxane, octamethyltrisiloxane, decamethyltetrasiloxane, tetradecamethylhexasiloxane, hexadecamethylheptasiloxane and dodecamethylpentasiloxane.

Volatile cyclic silicone oils that may be mentioned include hexamethylcyclotrisiloxane, octamethylcyclotetrasiloxane, decamethylcyclopentasiloxane, cyclohexasiloxane and dodecamethylcyclohexasiloxane, in particular cyclohexasiloxane.

Use may also be made of volatile fluoro oils, such as nonafluoromethoxybutane, decafluoropentane, tetradecafluorohexane, dodecafluoropentane, and mixtures thereof.

### Non-Volatile Oils

The term “non-volatile” refers to an oil whose vapour pressure at room temperature and atmospheric pressure is non-zero and is less than 10-3 mmHg (0.13 Pa).

The non-volatile oils may notably be chosen from non-volatile hydrocarbon-based, fluoro and/or silicone oils.

Non-volatile hydrocarbon-based oils that may notably be mentioned include:
hydrocarbon-based oils of animal origin,
linear or branched hydrocarbons of mineral or synthetic origin, such as petroleum jelly, polydecenes, hydrogenated polyisobutene such as Parleam, and squalane, and mixtures thereof,
non-volatile alkanes, preferably with a viscosity of less than 20 mPa·s at 20° C. measured with a Rheomat RM100® viscometer from Lamy Rheology. The term “non-volatile alkane” means a hydrocarbon-based cosmetic oil which is liquid at room temperature, notably having a vapour pressure at 20° C. of less than 0.01 kPa, according to the definition of a Volatile Organic Compound (VOC) of article 2 of European Council Directive 1999/13/EC of 11 March, 1992: “Any organic compound having, at a temperature of 293.15 K, a vapour pressure of 0.01 kPa or more”. In particular, the non-volatile alkanes comprise from 10 to 30 carbon atoms, in particular from 12 to 26 carbon atoms, and more particularly from 15 to 19 carbon atoms, and preferably a mixture of alkanes containing from 15 to 19 carbon atoms, for example the products sold under the references Emogreen L19 and Emosmart L19 from SEPPIC,
hydrocarbon-based oils of plant origin, such as glyceride triesters, which are generally fatty acid triesters of glycerol, the fatty acids of which may have varied chain lengths from 4 to 24 carbon atoms, it being possible for these chains to be saturated or unsaturated and linear or branched; these oils are notably wheatgerm oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame oil, marrow oil, rapeseed oil, blackcurrant oil, evening primrose oil, millet oil, barley oil, quinoa oil, rye oil, safflower oil, candlenut oil, passionflower oil and musk rose oil; or else caprylic/capric acid triglycerides, such as those sold by the company Stearinerie Dubois or those sold under the names Miglyol 810, 812 and 818 by the company Dynamit Nobel,
synthetic ethers containing from 10 to 40 carbon atoms, such as dicapryl ether,
synthetic esters, such as the oils of formula R₁COOR₂, in which R₁ represents a linear or branched fatty acid residue including from 1 to 40 carbon atoms and R₂ represents a hydrocarbon-based chain that is notably branched, containing from 1 to 40 carbon atoms, with the proviso that R₁+R₂ is greater than or equal to 10, for instance purcellin oil (cetostearyl octanoate), isopropyl myristate, isopropyl palmitate, alkyl benzoates containing between 12 and 15 carbon atoms, such as the product sold under the trade name Finsolv TN or Witconol TN by the company Witco or Tegosoft TN by the company Evonik Goldschmidt, 2-ethylphenyl benzoate, such as the commercial product sold under the name X-Tend 226 by the company ISP, isopropyl lanolate, hexyl laurate, diisopropyl adipate, isononyl isononanoate, oleyl erucate, 2-ethylhexyl palmitate, isostearyl isostearate, diisopropyl sebacate such as the product sold under the name Dub Dis by the company Stearinerie Dubois, alcohol or polyalcohol octanoates, decanoates or ricinoleates, such as propylene glycol dioctanoate; hydroxylated esters, such as isostearyl lactate, diisostearyl malate; and pentaerythritol esters; citrates, such as the ester of C₃-C₂₂ tricarboxylic acid and of C₁-C₆ alcohols having the INCI name Triethyl Citrate, for example the product sold under the name Citrofol Al Extra by the company Jungbunzlauer; tartrates, such as linear dialkyl tartrates containing 12 or 13 carbon atoms, for example those sold under the name Cosmacol ETI by the company Enichem Augusta Industriale, and also linear dialkyl tartrates containing between 14 and 15 carbon atoms, for example those sold under the name Cosmacol ETL by the same company, and acetates,
fatty amides, such as isopropyl N-lauroyl sarcosinate, for example the product sold under the trade name Eldew SL205 from Ajinomoto,
polyol esters and pentaerythritol esters, for instance dipentaerythrityl tetrahydroxystearate/tetraisostearate,
fatty alcohols that are liquid at room temperature, with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,
C₁₂-C₂₂ higher fatty acids, such as oleic acid, linoleic acid, linolenic acid, and mixtures thereof,
carbonates, such as dicaprylyl carbonate, for example the product sold under the name Cetiol CC by the company Cognis;
non-phenyl silicone oils, for instance caprylyl methicone, and
phenyl silicone oils, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates, dimethicones or phenyl trimethicone with a viscosity of less than or equal to 100 cSt, trimethylpentaphenyltrisiloxane, and mixtures thereof;
and also mixtures of these various oils.

### Other Fatty Substances

A fatty phase according to the invention may also comprise other fatty substances, mixed with or dissolved in an oil.

The other fatty substances that may be present in the oily phase are, for example:
fatty acids including from 8 to 30 carbon atoms, such as stearic acid, lauric acid, palmitic acid and oleic acid;
waxes, other than glyceryl trihydroxystearate, such as lanolin, beeswax, carnauba or candelilla wax, paraffin waxes, lignite waxes or microcrystalline waxes, ceresin or ozokerite, or synthetic waxes, such as polyethylene waxes or Fischer-Tropsch waxes;
silicone resins such as trifluoromethyl(C₁-C₄)alkyl dimethicone and trifluoropropyl dimethicone;
silicone elastomers such as the products sold under the name KSG by the company Shin-Etsu, under the names Trefil or BY29 by the company Dow Corning or under the name Gransil by the company Grant Industries;
a gum chosen from silicone gums (dimethiconol);
a pasty compound, such as polymeric or non-polymeric silicone compounds, esters of an oligomeric glycerol, arachidyl propionate, fatty acid triglycerides and derivatives thereof;
and mixtures thereof.

As fatty substances, a composition according to the invention may comprise at least one fatty alcohol wax. Such waxes may be chosen from lauryl alcohol, myristyl alcohol, cetyl alcohol, stearyl alcohol, arachidyl alcohol, behenyl alcohol, lignoceryl alcohol, ceryl alcohol, montanyl alcohol, myricyl alcohol, and mixtures thereof.

As fatty substance, a composition according to the invention may in particular comprise at least one butter, in particular a plant butter.

The plant butters that are suitable for use in the invention are preferably chosen from the group comprising avocado butter, cocoa butter, shea butter, kokum butter, mango butter, murumuru butter, coconut butter, apricot kernel butter, sal butter and urukum butter, and mixtures thereof, and in particular is shea butter.

These fatty substances may be chosen in a varied manner by a person skilled in the art in order to prepare a composition having the desired properties, for example in terms of consistency or texture.

According to a preferred embodiment, a composition according to the invention comprises at least one non-volatile oil, preferably at least one non-volatile hydrocarbon-based oil, in particular chosen from linear or branched hydrocarbons of mineral or synthetic origin, non-volatile alkanes, hydrocarbon-based oils of plant origin, synthetic esters, fatty amides, carbonates, and also mixtures of these various oils.

In particular, such non-volatile hydrocarbon-based oils may be present in a composition according to the invention in a content ranging from 2.0% to 20.0% by weight and preferably from 2.0% to 15.0% by weight relative to the total weight of the composition.

According to a preferred embodiment, a composition according to the invention contains at least one oil chosen from squalane, mixtures of alkanes containing from 15 to 19 carbon atoms, caprylic/capric acid triglycerides, alkyl benzoates containing between 12 and 15 carbon atoms, diisopropyl adipate, 2-ethylhexyl palmitate, diisopropyl sebacate, the ester of C₃-C₂₂ tricarboxylic acid and of C₁-C₆ alcohols having the INCI name Triethyl Citrate, isopropyl N-lauroyl sarcosinate, dicaprylyl carbonate, and mixtures thereof.

Preferably, a composition according to the invention comprises less than 2.0% by weight of silicone oil(s), in particular less than 1.0% by weight of silicone oil(s), preferably less than 0.5% by weight of silicone oil(s), relative to the total weight of the composition, and more preferentially is free of silicone oil(s).

Preferably, a composition according to the invention comprises a fatty phase containing at least one fatty substance.

According to a preferred embodiment, a composition according to the invention comprises at least one non-volatile hydrocarbon-based oil, and preferably at least one non-volatile ester oil.

According to a preferred embodiment, a composition according to the invention also comprises at least one solid fatty substance, in particular a fatty acid including from 8 to 30 carbon atoms, and more preferentially stearic acid.

### Additional UV-Screening Agents

A composition according to the invention may comprise at least one additional UV-screening agent other than the merocyanines of formula (I) described previously.

In particular, a composition according to the invention may comprise at least one compound for screening out UVA and/or UVB other than the merocyanines of formula (I) described previously.

The compositions according to the invention may thus also contain one or more additional UV-screening agents chosen from hydrophilic, lipophilic or insoluble organic UV-screening agents and/or one or more mineral pigments. It will preferentially consist of at least one hydrophilic, lipophilic or insoluble organic UV-screening agent.

The term “hydrophilic UV screening agent” means any organic or inorganic cosmetic or dermatological compound which screens out UV radiation, which is liable to be fully dissolved in molecular form in a liquid aqueous phase or else which can be solubilized in colloidal form (for example in micellar form) in a liquid aqueous phase. The term “lipophilic screening agent” means any cosmetic or dermatological organic or inorganic compound for screening out UV radiation, which is liable to be fully dissolved in molecular form in a liquid fatty phase or else which can be solubilized in colloidal form (for example in micellar form) in a liquid fatty phase.

The term “insoluble UV-screening agent” means any cosmetic or dermatological organic or inorganic compound for screening out UV radiation, which has a solubility in water of less than 0.5% by weight and a solubility of less than 0.5% by weight in the majority of organic solvents such as liquid paraffin, fatty alcohol benzoates and fatty acid triglycerides, for example Miglyol 812® sold by the company Dynamit Nobel. This solubility, determined at 70° C., is defined as the amount of product in solution in the solvent at equilibrium with an excess of solid in suspension after returning to room temperature. It may be readily evaluated in the laboratory. The additional organic UV-screening agents are notably chosen from cinnamic compounds; anthranilate compounds; salicylic compounds; dibenzoylmethane compounds; benzylidenecamphor compounds; benzophenone compounds; β,β-diphenylacrylate compounds; triazine compounds; benzotriazole compounds; benzalmalonate compounds, notably those cited in patent U.S. Pat. No. 5,624,663; benzimidazole derivatives; imidazoline compounds; bis-benzazolyl compounds, such as described in patents EP 669 323 and U.S. Pat. No. 2,463,264; p-aminobenzoic (PABA) compounds; methylenebis(hydroxyphenylbenzotriazole) compounds, such as described in patent applications U.S. Pat. Nos. 5,237,071, 5,166,355, GB 2 303 549, DE 197 26 184 and EP 893 119; benzoxazole compounds, such as described in patent applications EP 0 832 642, EP 1 027 883, EP 1 300 137 and DE 101 62 844; screening polymers and screening silicones, such as those notably described in patent application WO 93/04665; a-alkylstyrene-based dimers, such as those described in patent application DE 198 55 649; 4,4-diarylbutadiene compounds, such as described in patent applications EP 0 967 200, DE 197 46 654, DE 197 55 649, EP-A-1 008 586, EP 1 133 980 and EP 133 981, and mixtures thereof.

As examples of organic photoprotective agents, mention may be made of those denoted hereinbelow under their INCI name:

### Cinnamic Compounds:

Ethylhexyl Methoxycinnamate notably sold under the trade name Parsol MCX® by DSM Nutritional Products,
Isopropyl Methoxycinnamate,
Isoamyl p-methoxycinnamate sold under the trade name Neo Heliopan E 1000® by Symrise,
DEA Methoxycinnamate,
Diisopropyl Methylcinnamate,
Glyceryl Ethylhexanoate Dimethoxycinnamate.

### Dibenzoylmethane Compounds:

Butyl Methoxydibenzoylmethane, notably sold under the trade name Parsol 1789® by DSM Nutritional Products,
Isopropyl Dibenzoylmethane.

### Para-Aminobenzoic Compounds:

PABA,
Ethyl PABA,
Ethyl Dihydroxypropyl PABA,
Ethylhexyl Dimethyl PABA notably sold under the name Escalol 507® by ISP,
Glyceryl PABA,
PEG-25 PABA sold under the name Uvinul P25® by BASF.

### Salicylic Compounds:

Homosalate sold under the name Eusolex HMS® by Rona/EM Industries,
Ethylhexyl Salicylate sold under the name Neo Heliopan OS® by Symrise,
Dipropylene glycol Salicylate sold under the name Dipsal® by Scher,
TEA Salicylate, sold under the name Neo Heliopan TS® by Symrise.

### β,β-Diphenylacrylate Compounds:

Octocrylene notably sold under the trade name Uvinul N539® by BASF,
Etocrylene, notably sold under the trade name Uvinul N35® by BASF.

Benzophenone compounds:
Benzophenone-1, sold under the trade name Uvinul 400® by BASF,
Benzophenone-2, sold under the trade name Uvinul D 50® by BASF,
Benzophenone-3 or Oxybenzone, sold under the trade name Uvinul M 40® by BASF,
Benzophenone-4, sold under the trade name Uvinul MS 40® by BASF,
Benzophenone-5,
Benzophenone-6, sold under the trade name Helisorb 11® by Norquay,
Benzophenone-8, sold under the trade name Spectra-Sorb UV-24® by American Cyanamid,
Benzophenone-9, sold under the trade name Uvinul DS 49® by BASF,
Benzophenone-12,
n-Hexyl 2-(4-diethylamino-2-hydroxybenzoyl)benzoate, sold under the trade name Uvinul A Plus® or, as a mixture with octyl methoxycinnamate, under the trade name Uvinul A Plus B® by the company BASF,
1,1′-(1,4-Piperazinediyl)bis[1-[2-[4-(diethylamino)-2-hydroxybenzoyl]phenyl]methanone] (CAS 919803-06-8), as described in patent application WO 2007/071584; this compound advantageously being used in micronized form (mean size of 0.02 to 2 μm), which may be obtained, for example, according to the micronization process described in patent applications GB-A-2 303 549 and EP-A-893 119, and notably in the form of an aqueous dispersion.

### Benzylidenecamphor Compounds:

3-Benzylidenecamphor manufactured under the name Mexoryl SD® by Chimex,
4-Methylbenzylidenecamphor sold under the name Eusolex 6300® by Merck,
Benzylidenecamphorsulfonic acid manufactured under the name Mexoryl SL® by Chimex,
Camphor Benzalkonium Methosulfate manufactured under the name Mexoryl SO® by Chimex,
Terephthalylidene Dicamphor Sulfonic Acid manufactured under the name Mexoryl SX® by Chimex,
Polyacrylamidomethyl Benzylidene Camphor, manufactured under the name Mexoryl SW® by Chimex.

### Phenylbenzimidazole Compounds:

Phenylbenzimidazole Sulfonic Acid, notably sold under the trade name Eusolex 232® by Merck.

### Bis-Benzazolyl Compounds:

Disodium Phenyl Dibenzimidazole Tetrasulfonate, sold under the trade name Neo Heliopan AP® by Haarmann and Reimer.

### Phenylbenzotriazole Compounds:

Drometrizole Trisiloxane, sold under the name Silatrizole® by Rhodia Chimie.

### Methylenebis(Hydroxyphenylbenzotriazole) Compounds:

Methylene bis-Benzotriazolyl Tetramethylbutylphenol, notably in solid form, such as the product sold under the trade name Mixxim BB/100® by Fairmount Chemical, or in the form of an aqueous dispersion of micronized particles with a mean particle size ranging from 0.01 to 5 μm, more preferentially from 0.01 to 2 μm and more particularly from 0.020 to 2 μm, with at least one alkyl polyglycoside surfactant having the structure CₙH₂ₙ₊₁O(C₆H₁₀O₅)ₓH, in which n is an integer from 8 to 16 and x is the mean degree of polymerization of the (C₆H₁₀O₅) unit and ranges from 1.4 to 1.6, as described in patent GB-A-2 303 549, notably sold under the trade name Tinosorb M® by BASF, or in the form of an aqueous dispersion of micronized particles with a mean particle size ranging from 0.02 to 2 μm, more preferentially from 0.01 to 1.5 μm and more particularly from 0.02 to 1 μm, in the presence of at least one polyglyceryl mono(C₈-C₂₀)alkyl ester with a degree of glycerol polymerization of at least 5, such as the aqueous dispersions described in patent application WO 2009/063392.

### Triazine Compounds:

3,3′-(1,4-Phenylene)bis(5,6-diphenyl-1,2,4-triazine), having the INCI name Phenylene Bis-Diphenyltriazine, and having the following chemical formula:

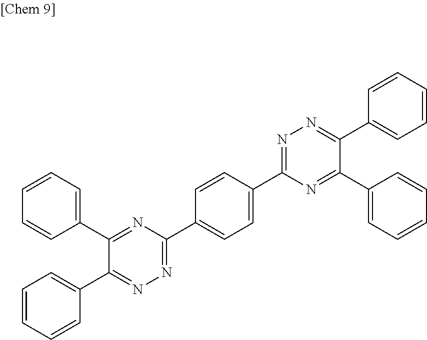 [SMILES: c1ccc(-c2nnc(-c3ccc(-c4nnc(-c5ccccc5)c(-c5ccccc5)n4)cc3)nc2-c2ccccc2)cc1]

Bis-Ethylhexyloxyphenol Methoxyphenyl Triazine, sold under the trade name Tinosorb S® by BASF,
Ethylhexyl Triazone, notably sold under the trade name Uvinul T 150® by BASF,
Diethylhexyl Butamido Triazone, sold under the trade name Uvasorb HEB® by Sigma 3V,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4′-aminobenzalmalonate)-6-(n-butyl 4′-aminobenzoate)-s-triazine,
symmetrical triazine screening agents substituted with naphthalenyl groups or polyphenyl groups described in patent U.S. Pat. No. 6,225,467, patent application WO 2004/085412 (see compounds 6 and 9) or the document “Symmetrical Triazine Derivatives”, IP.COM IPCOM000031257 Journal, INC West Henrietta, NY, US (20 Sep. 2004), notably 2,4,6-tris(diphenyl)triazine and 2,4,6-tris(terphenyl)triazine, which is also mentioned in patent applications WO 06/035000, WO 06/034982, WO 06/034991, WO 06/035007, WO 2006/034992 and WO 2006/034985, these compounds advantageously being used in micronized form (mean particle size of 0.02 to 3 μm), which can be obtained, for example, according to the micronization process described in patent applications GB-A-2 303 549 and EP-A-893 119, and notably in aqueous dispersion form,
silicone triazines substituted with two aminobenzoate groups, as described in patent EP 0 841 341, in particular 2,4-bis(n-butyl 4′-aminobenzalmalonate)-6-[(3-{1,3,3,3-tetramethyl-1-[(trimethylsilyl)oxy]disiloxanyl}propyl)amino]-s-triazine.

### Anthranilic Compounds:

Menthyl Anthranilate, sold under the trade name Neo Heliopan MA® by Symrise.

### Imidazoline Compounds:

Ethylhexyl dimethoxybenzylidene dioxoimidazoline propionate.

### Benzalmalonate Compounds:

Polyorganosiloxane comprising benzalmalonate functions, such as Polysilicone-15, sold under the trade name Parsol SLX® by Hoffmann-LaRoche.

### 4,4-Diarylbutadiene Compounds:

1,1-Dicarboxy(2,2′-dimethylpropyl)-4,4-diphenylbutadiene.

### Benzoxazole Compounds:

2,4-Bis[5-1(dimethylpropyl)benzoxazol-2-yl-(4-phenyl)imino]-6-(2-ethylhexyl)imino-1,3,5-triazine sold under the name Uvasorb K2A® by Sigma 3V.

The preferential organic screening agents are chosen from:
Ethylhexyl methoxycinnamate,
Ethylhexyl salicylate,
Homosalate,
Butylmethoxydibenzoylmethane,
Octocrylene,
Phenylbenzimidazolesulfonic acid,
Benzophenone-3,
Benzophenone-4,
Benzophenone-5,
n-Hexyl 2-(4-diethylamino-2-hydroxybenzoyl)benzoate,
4-Methylbenzylidenecamphor,
Terephthalylidenedicamphorsulfonic acid,
Disodium phenyldibenzimidazoletetrasulfonate,
Methylenebis(benzotriazolyl)tetramethylbutylphenol,
Bis(ethylhexyloxyphenol)methoxyphenyltriazine,
Ethylhexyl triazone,
Diethylhexylbutamidotriazone,
2,4,6-Tris(dineopentyl 4′-aminobenzalmalonate)-s-triazine,
2,4,6-Tris(diisobutyl 4′-aminobenzalmalonate)-s-triazine,
2,4-Bis(n-butyl 4′-aminobenzoate)-6-(aminopropyltrisiloxane)-s-triazine,
2,4-Bis(dineopentyl 4′-aminobenzalmalonate)-6-(n-butyl 4′-aminobenzoate)-s-triazine,
2,4-Bis(n-butyl 4′-aminobenzalmalonate)-6-[(3-{1,3,3,3-tetramethyl-1-[(trimethylsilyloxy]disiloxanyl}propyl)amino]-s-triazine,
2,4,6-Tris(diphenyl)triazine,
2,4,6-Tris(terphenyl)triazine,
Drometrizole trisiloxane,
Polysilicone-15,
1,1-Dicarboxy(2,2′-dimethylpropyl)-4,4-diphenylbutadiene,
2,4-Bis[5-1(dimethylpropyl)benzoxazol-2-yl(4-phenyl)imino]-6-(2-ethylhexyl)imino-1,3,5-triazine,
and mixtures thereof.

The particularly preferred organic screening agents are chosen from:
Ethylhexyl salicylate,
Homosalate,
Butylmethoxydibenzoylmethane,
Octocrylene,
n-Hexyl 2-(4-diethylamino-2-hydroxybenzoyl)benzoate,
Terephthalylidenedicamphorsulfonic acid,
Bis(ethylhexyloxyphenol)methoxyphenyltriazine,
Ethylhexyl triazone,
Diethylhexyl butamido triazone,
2,4-Bis(n-Butyl 4′-aminobenzalmalonate)-6-[(3-{1,3,3,3-tetramethyl-1-[(trimethylsilyloxy]disiloxanyl}propyl)amino]-s-triazine,
Drometrizole trisiloxane,
and mixtures thereof.

The inorganic UV-screening agents used in accordance with the present invention are metal oxide pigments. More preferentially, the inorganic UV-screening agents of the invention are metal oxide particles with a mean elementary particle size of less than or equal to 0.5 μm, more preferentially between 0.005 and 0.5 μm, even more preferentially between 0.01 and 0.2 μm, better still between 0.01 and 0.1 μm and more particularly between 0.015 and 0.05 μm.

They may notably be chosen from titanium oxide, zinc oxide, iron oxide, zirconium oxide and cerium oxide, or mixtures thereof.

Such coated or uncoated metal oxide pigments are described in particular in patent application EP-A-0 518 773. As commercial pigments, mention may be made of the products sold by the companies Sachtleben Pigments, Tayca, Merck and Degussa. The metal oxide pigments may be coated or uncoated.

The coated pigments are pigments that have undergone one or more surface treatments of chemical, electronic, mechanochemical and/or mechanical nature with compounds such as amino acids, beeswax, fatty acids, fatty alcohols, anionic surfactants, lecithins, sodium, potassium, zinc, iron or aluminium salts of fatty acids, metal alkoxides (of titanium or aluminium), polyethylene, silicones, proteins (collagen, elastin), alkanolamines, silicon oxides, metal oxides or sodium hexametaphosphate.

The coated pigments are more particularly titanium oxides that have been coated:
with silica, such as the product Sunveil® from the company Ikeda,
with silica and iron oxide, such as the product Sunveil F® from the company Ikeda,
with silica and alumina, such as the products Microtitanium Dioxide MT 500 SA® and Microtitanium Dioxide MT 100 SA® from the company Tayca and Tioveil from the company Tioxide,
with alumina, such as the products Tipaque TTO-55 (B)® and Tipaque TTO-55 (A)® from the company Ishihara and UVT 14/4 from the company Sachtleben Pigments,
with alumina and aluminium stearate, such as the products Microtitanium Dioxide MT 100 T®, MT 100 TX®, MT 100 Z® and MT-01® from the company Tayca, the products Solaveil CT-10 W® and Solaveil CT 100® from the company Uniqema and the product Eusolex T-AVO® from the company Merck,
with silica, alumina and alginic acid, such as the product MT-100 AQ® from the company Tayca,
with alumina and aluminium laurate, such as the product Microtitanium Dioxide MT 100 S® from the company Tayca,
with iron oxide and iron stearate, such as the product Microtitanium Dioxide MT 100 F® from the company Tayca,
with zinc oxide and zinc stearate, such as the product BR 351® from the company Tayca,
with silica and alumina and treated with a silicone, such as the products Microtitanium Dioxide MT 600 SAS®, Microtitanium Dioxide MT 500 SAS® or Microtitanium Dioxide MT 100 SAS® from the company Tayca,
with silica, alumina and aluminium stearate and treated with a silicone, such as the product STT-30-DS® from the company Titan Kogyo,
with silica and treated with a silicone, such as the product UV-Titan X 195® from the company Sachtleben Pigments,
with alumina and treated with a silicone, such as the products Tipaque TTO-55 (S)® from the company Ishihara or UV Titan M 262® from the company Sachtleben Pigments,
with triethanolamine, such as the product STT-65-S from the company Titan Kogyo,
with stearic acid, such as the product Tipaque TTO-55 (C)® from the company Ishihara,
with sodium hexametaphosphate, such as the product Microtitanium Dioxide MT 150 W® from the company Tayca,
TiO₂ treated with octyltrimethylsilane, sold under the trade name T 805® by the company Degussa Silices,
TiO₂ treated with a polydimethylsiloxane, sold under the trade name 70250 Cardre UF TiO₂SI₃® by the company Cardre,
anatase/rutile TiO₂ treated with a polydimethylhydrosiloxane, sold under the trade name Microtitanium Dioxide USP Grade Hydrophobic® by the company Color Techniques.
TiO₂ coated with triethylhexanoin, aluminium stearate and alumina, sold under the trade name Solaveil CT-200-LQ-(WD) by Croda,
TiO₂ coated with aluminium stearate, with alumina and with silicone, sold under the trade name Solaveil CT-12W-LQ-(WD) by Croda,
TiO₂ coated with lauroyl lysine, sold by Daito Kasei Kogyo under the name LL 5 Titanium Dioxide CR 50,
TiO₂ coated with C₉-C₁₅ fluoro alcohol phosphate and with aluminium hydroxide, sold by Daito Kasei Kogyo under the name PFX-5 TiO₂ CR-50.

Mention may also be made of TiO₂ pigments doped with at least one transition metal such as iron, zinc or manganese and more particularly manganese. Preferably, said doped pigments are in the form of an oily dispersion. The oil present in the oily dispersion is preferably chosen from triglycerides including those of capric/caprylic acids. The oily dispersion of titanium oxide particles may also include one or more dispersants, for instance a sorbitan ester, for instance sorbitan isostearate, or a polyoxyalkylenated fatty acid ester of glycerol, for instance TRI-PPG-3 myristyl ether citrate and polyglyceryl-3 polyricinoleate. Preferably, the oily dispersion of titanium oxide particles includes at least one dispersant chosen from polyoxyalkylenated fatty acid esters of glycerol. Mention may be made more particularly of the oily dispersion of TiO₂ particles doped with manganese in capric/caprylic acid triglyceride in the presence of TRI-PPG-3 myristyl ether citrate and polyglyceryl-3 polyricinoleate and sorbitan isostearate having the INCI name: Titanium Dioxide (and) Tri-PPG-3 Myristyl Ether Citrate (and) Polyglyceryl-3 Ricinoleate (and) Sorbitan Isostearate, such as the product sold under the trade name Optisol TD50® by the company Croda.

The uncoated titanium oxide pigments are sold, for example, by the company Tayca under the trade names Microtitanium Dioxide MT 500 B or Microtitanium Dioxide MT 600 B®, by the company Degussa under the name P 25, by the company Wacker under the name Transparent titanium oxide PW®, by the company Miyoshi Kasei under the name UFTR®, by the company Tomen under the name ITS® and by the company Tioxide under the name Tioveil AQ.

The uncoated zinc oxide pigments are, for example:
those sold under the name Z-Cote by the company Sunsmart;
those sold under the name Nanox® by the company Elementis;
those sold under the name Nanogard WCD 2025® by the company Nanophase Technologies.

The coated zinc oxide pigments are, for example:
those sold under the name Zinc Oxide CS-5® by the company Toshibi (ZnO coated with polymethylhydrogenosiloxane);
those sold under the name Nanogard Zinc Oxide FN® by the company Nanophase Technologies (as a 40% dispersion in Finsolv TN®, C₁₂-C₁₅ alkyl benzoates);
those sold under the name Daitopersion Zn-30® and Daitopersion Zn-50® by the company Daito (dispersions in oxyethylenated polydimethylsiloxane/cyclopolymethylsiloxane, containing 30% or 50% of zinc oxides coated with silica and polymethylhydrogenosiloxane);
those sold under the name NFD Ultrafine ZnO® by the company Daikin (ZnO coated with perfluoroalkyl phosphate and copolymer based on perfluoroalkylethyl in dispersion in cyclopentasiloxane);
those sold under the name SPD-Z1® by the company Shin-Etsu (ZnO coated with silicone-grafted acrylic polymer, dispersed in cyclodimethylsiloxane);
those sold under the name Escalol Z100® by the company ISP (alumina-treated ZnO dispersed in an ethylhexyl methoxycinnamate/PVP-hexadecene copolymer/methicone mixture);
those sold under the name Fuji ZnO-SMS-10® by the company Fuji Pigment (ZnO coated with silica and polymethylsilsesquioxane);
those sold under the name Nanox Gel TN® by the company Elementis (ZnO dispersed at 55% in C₁₂-C₁₅ alkyl benzoate with hydroxystearic acid polycondensate).

The uncoated cerium oxide pigments may, for example, be those sold under the name Colloidal Cerium Oxide® by the company Rhône-Poulenc.

The uncoated iron oxide pigments are sold, for example, by the company Arnaud under the names Nanogard WCD 2002® (FE 45B®), Nanogard Iron FE 45 BL AQ, Nanogard FE 45R AQ® and Nanogard WCD 2006® (FE 45R®) or by the company Mitsubishi under the name TY-220®.

The coated iron oxide pigments are sold, for example, by the company Arnaud under the names Nanogard WCD 2008 (FE 45B FN®), Nanogard WCD 2009® (FE 45B 556®), Nanogard FE 45 BL 345® and Nanogard FE 45 BL® or by the company BASF under the name Transparent Iron Oxide®.

Mention may also be made of mixtures of metal oxides, notably of titanium dioxide and of cerium dioxide, including the equal-weight mixture of titanium dioxide and cerium dioxide coated with silica, sold by the company Ikeda under the name Sunveil A®, and also the mixture of titanium dioxide and zinc dioxide coated with alumina, silica and silicone, such as the product M 261® sold by the company Sachtleben Pigments, or coated with alumina, silica and glycerol, such as the product M 211® sold by the company Sachtleben Pigments.

According to the invention, coated or uncoated titanium oxide pigments are particularly preferred.

According to a particular embodiment, the composition comprises one or more lipophilic UV-screening agents.

According to a preferred embodiment, the composition comprises one or more UV-screening agents chosen from lipophilic UVA-screening agents and/or lipophilic UVB-screening agents.

More preferentially, the composition comprises one or more UV-screening agents chosen from dibenzoylmethane derivatives, salicylic derivatives, β,β′-diphenyl acrylate derivatives, triazine derivatives and benzylidenecamphor derivatives.

According to a particularly preferred embodiment, the composition comprises one or more UV-screening agents chosen from Terephthalylidene Dicamphor Sulfonic Acid, Butyl Methoxydibenzoylmethane, Homosalate, Ethylhexyl Salicylate, Octocrylene, Ethylhexyl Triazone, and mixtures thereof.

According to one embodiment, the amount of the organic UV-screening agent(s) present in the composition according to the invention may range from 0.5% to 10% by weight, relative to the total weight of the composition. It ranges for example from 1% to 10% by weight, or else for example from 5% to 10% by weight, relative to the total weight of the composition.

According to a particular embodiment, the composition according to the invention comprises at least one mineral UV-screening agent.

According to a particular embodiment, the amount of the mineral UV-screening agent(s) present in the composition according to the invention may range from 0.01% to 10% by weight relative to the total weight of the composition. It ranges, for example, from 1% to 5% by weight, relative to the total weight of the composition.

According to a particular embodiment, the composition according to the invention also comprises one or more organic UV-screening agents and one or more mineral UV-screening agents.

According to one particular embodiment, the composition according to the invention also comprises a combination of UV-screening agents as described in patent FR 2 977 490, patent application WO 2013/004777 or patent application US 2014/0134120.

### Surfactants

According to a particular embodiment, the composition according to the invention may also comprise at least one surfactant.

The surfactants may be chosen from nonionic, anionic, cationic and amphoteric surfactants, and mixtures thereof. Reference may be made to the Kirk-Othmer Encyclopedia of Chemical Technology, volume 22, pages 333-432, 3rd Edition, 1979, Wiley, for the definition of the emulsifying properties and functions of surfactants, in particular pages 347-377 of this reference, for anionic, amphoteric and nonionic surfactants.

### Nonionic Surfactant

Preferably, the composition according to the invention comprises at least one nonionic surfactant.

The nonionic surfactants may notably be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, glyceryl esters, optionally polyoxyethylenated alkyl and polyalkyl esters of glycerol, and optionally polyoxyethylenated alkyl and polyalkyl ethers of glycerol, gemini surfactants, cetyl alcohol, stearyl alcohol, and mixtures thereof.

As glyceryl esters, mention may notably be made of C₁₆-C₂₂ fatty acid esters of glycerol, in particular glyceryl esters of a fatty acid containing 18 carbon atoms, and polyglyceryl esters of a fatty acid containing from 8 to 12 carbon atoms.

Glyceryl esters of a C₁₈ fatty acid that may notably be mentioned include glyceryl stearate (glyceryl monostearate, distearate and/ortristearate) (CTFA name: glyceryl stearate) or glyceryl ricinoleate, or mixtures thereof.

As fatty acid ester of glycerol, mention may be made of mixtures based on glyceryl stearate, such as the mixture of glyceryl stearate and polyethylene glycol 100 OE monostearate, and in particular the one comprising a 50/50 mixture, sold under the name Arlacel 165® by the company Croda, or else the product containing glyceryl stearate (glyceryl mono-distearate) and potassium stearate, sold under the name Tegin® by the company Goldschmidt (CTFA name: glyceryl stearate SE).

Preferably, a composition according to the invention comprises at least one surfactant, in particular a nonionic surfactant, and preferably a surfactant chosen from glyceryl esters.

### Anionic Surfactant

The anionic surfactants may be chosen from alkyl ether sulfates, carboxylates, amino acid derivatives, sulfonates, isethionates, taurates, sulfosuccinates, alkyl sulfoacetates, phosphates and alkyl phosphates, polypeptides, metal salts of C₁₀-C₃₀ and notably C₁₆-C₂₅ fatty acids, in particular metal stearates and behenates, alkali metal salts of cetyl phosphate, and mixtures thereof.

As alkali metal salts of cetyl phosphate, mention may notably be made of potassium cetyl phosphate. Use may notably be made of the monopotassium salt of monocetyl phosphate (INCI name: potassium cetyl phosphate) sold under the name Amphisol K by the company DSM Nutritional Products.

### Cationic Surfactant

The cationic surfactants may be chosen from alkylimidazolidiniums, such as isostearyl ethylimidonium ethosulfate, ammonium salts such as (C₁₂₋₃₀-alkyl)tri(C₁₋₄-alkyl)ammonium halides such as N,N,N-trimethyl-1-docosanaminium chloride (or behentrimonium chloride).

### Amphoteric Surfactant

The compositions according to the invention may also contain one or more amphoteric surfactants, for instance N-acylamino acids such as N-alkyl aminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

### Silicone Surfactant

The composition may also comprise at least one silicone surfactant. By way of example, as nonionic surfactants with an HLB of greater than or equal to 8 at 25° C., used alone or as a mixture, mention may be made of dimethicone copolyol or dimethicone copolyol benzoate, and as nonionic surfactants with an HLB of less than 8 at 25° C., used alone or as a mixture, mention may be made of a cyclomethicone/dimethicone copolyol mixture.

Preferably, a composition according to the invention comprises less than 2% by weight of silicone surfactant(s), in particular less than 1% by weight of silicone surfactant(s), preferably less than 0.5% by weight of silicone surfactant(s) and more preferentially is free of silicone surfactant(s).

The surfactant(s) may be present in a composition according to the invention in a proportion ranging from 0.5% to 10% by weight and preferably from 0.5% to 5% by weight, relative to the total weight of the composition.

### Gelling Agent/Thickener

Depending on the fluidity of the composition that it is desired to obtain, one or more thickeners and/or gelling agents, which are notably hydrophilic, that is to say water-soluble or water-dispersible, may be incorporated into the composition.

Examples of hydrophilic gelling agents that may be mentioned include modified or unmodified carboxyvinyl polymers, such as the products sold under the names Carbopol (CTFA name: carbomer) and Pemulen (CTFA name: Acrylates/C10-30 alkyl acrylate crosspolymer) by the company Goodrich; polyacrylamides; optionally crosslinked and/or neutralized 2-acrylamido-2-methylpropanesulfonic acid polymers and copolymers, for instance the poly(2-acrylamido-2-methylpropanesulfonic acid) sold by the company Hoechst under the name Hostacerin AMPS (CTFA name: Ammonium polyacryldimethyltauramide); crosslinked anionic copolymers of acrylamide and of AMPS, which are in the form of a W/O emulsion, such as those sold under the name Sepigel 305 (CTFA name: Polyacrylamide/C13-14 Isoparaffin/Laureth-7) and under the name Simulgel 600 (CTFA name: Acrylamide/Sodium acryloyldimethyltaurate copolymer/Isohexadecane/Polysorbate 80) by the company SEPPIC; polysaccharide biopolymers, for instance xanthan gum, guar gum, alginates and modified celluloses; and mixtures thereof. The amount of gelling agents depends on the desired objective.

According to one embodiment, the amount of gelling agents ranges for example from 0.01% to 10% and for example from 0.05% to 5% by weight, relative to the total weight of the composition.

### Fillers

According to a particular embodiment, a composition according to the invention may also comprise one or more fillers, in particular chosen from those conventionally used in care and/or makeup compositions.

For the purposes of the present invention, the term “fillers” is understood to mean colourless or white, mineral or organic, natural or synthetic solid particles of any form, which are in a form that is insoluble and dispersed in the medium of the composition.

Needless to say, these fillers are used in appropriate contents and under appropriate conditions so as not to be detrimental to the properties of the composition.

These fillers make it possible to give the composition containing them softness, a matt effect and uniformity. In addition, these fillers advantageously make it possible to combat various attacking factors such as sebum or sweat.

By way of illustration of these fillers, mention may be made of talc, mica, silica, kaolin, poly-β-alanine powder and polyethylene powder, tetrafluoroethylene polymer (Teflon®) powders, lauroyllysine, starch, boron nitride, hollow polymer microspheres such as those of polyvinylidene chloride/acrylonitrile, for instance Expancel® (Nobel Industrie), acrylic acid copolymer microspheres, silicone resin microbeads (for example Tospearls® from Toshiba), polyorganosiloxane elastomer particles, precipitated calcium carbonate, magnesium carbonate, magnesium hydrogen carbonate, hydroxyapatite, barium sulfate, aluminium oxides, polyurethane powders, composite fillers, hollow silica microspheres, and glass or ceramic microcapsules. Use may also be made of particles that are in the form of hollow sphere portions, as described in patent applications JP-2003 128 788 and JP-2000 191 789.

### Dyestuffs

A composition according to the invention may also comprise at least one particulate or non-particulate, water-soluble or water-insoluble dyestuff, preferably in a proportion of at least 0.0001% by weight relative to the total weight of the composition.

For obvious reasons, this amount is liable to vary significantly with regard to the desired intensity of the colour effect and to the colour intensity provided by the dyestuffs under consideration, and its adjustment clearly falls within the competence of a person skilled in the art.

As stated above, the dyestuffs that are suitable for use in the invention may be water-soluble, but may also be liposoluble.

### Water-Soluble Dyes

For the purposes of the invention, the term “water-soluble dyestuff” means any natural or synthetic, generally organic compound, which is soluble in an aqueous phase or water-miscible solvents and which is capable of imparting colour.

As water-soluble dyes that are suitable for use in the invention, mention may be made notably of synthetic or natural water-soluble dyes, for instance FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, FDC Blue 1, betanine (beetroot), carmine, copper chlorophyllin, methylene blue, anthocyanins (enocianin, black carrot, hibiscus, elder), caramel and riboflavin.

The water-soluble dyes are, for example, beetroot juice and caramel.

According to a preferred embodiment, the composition according to the invention comprises at least one water-soluble dye.

The water-soluble dyes may be present in a proportion of from 0.0001% to 2% by weight relative to the total weight of the composition containing them.

### Liposoluble Dyes

For the purposes of the invention, the term “liposoluble dyestuff” means any natural or synthetic, generally organic compound, which is soluble in an oily phase or in solvents that are miscible with a fatty substance, and which is capable of imparting colour.

As liposoluble dyes that are suitable for use in the invention, mention may notably be made of synthetic or natural liposoluble dyes, for instance DC Red 17, DC Red 21, DC Red 27, DC Green 6, DC Yellow 11, DC Violet 2, DC Orange 5, Sudan red, carotenes (β-carotene, lycopene), xanthophylls (capsanthin, capsorubin, lutein), palm oil, Sudan brown, quinoline yellow, annatto and curcumin.

The colouring particulate materials may be present in a proportion of from 0.0001% to 10% by weight relative to the total weight of the composition containing them.

They may notably be pigments, nacres and/or particles with metallic tints.

### Pigments

The term “pigments” should be understood as meaning white or coloured, mineral or organic particles that are insoluble in an aqueous solution, which are intended to colour and/or opacify the composition containing them.

The pigments may be white or coloured, and mineral and/or organic.

As mineral pigments, mention may be made of titanium oxide, titanium dioxide, zirconium oxide, zirconium dioxide, cerium oxide or cerium dioxide and also zinc oxide, iron oxide or chromium oxide, ferric blue, manganese violet, ultramarine blue and chromium hydrate, and mixtures thereof.

It may also be a pigment having a structure that may be, for example, of sericite/brown iron oxide/titanium dioxide/silica type. Such a pigment is sold, for example, under the reference Coverleaf NS or JS by the company Chemicals and Catalysts, and has a contrast ratio in the region of 30.

They may also be pigments having a structure that may be, for example, of silica microsphere type containing iron oxide. An example of a pigment having this structure is the product sold by the company Miyoshi under the reference PC Ball PC-LL-100 P, this pigment consisting of silica microspheres containing yellow iron oxide.

Advantageously, the pigments may be iron oxides and/or titanium dioxides.

According to a particular embodiment, a composition according to the invention comprises less than 1% of pigments.

### Nacres

The term “nacres” should be understood as meaning coloured particles of any form, which may or may not be iridescent, notably produced by certain molluscs in their shell, or alternatively synthesized, and which have a colour effect via optical interference.

The nacres may be chosen from nacreous pigments such as titanium mica coated with an iron oxide, titanium mica coated with bismuth oxychloride, titanium mica coated with chromium oxide, titanium mica coated with an organic dye and also nacreous pigments based on bismuth oxychloride. They may also be mica particles, at the surface of which are superposed at least two successive layers of metal oxides and/or of organic dyestuffs.

Examples of nacres that may also be mentioned include natural mica covered with titanium oxide, with iron oxide, with natural pigment or with bismuth oxychloride.

Among the nacres available on the market, mention may be made of the Timica, Flamenco and Duochrome nacres (based on mica) sold by the company Engelhard, the Timiron nacres sold by the company Merck, the Prestige mica-based nacres, sold by the company Eckart, and the Sunshine synthetic mica-based nacres, sold by the company Sun Chemical.

The nacres may more particularly have a yellow, pink, red, bronze, orangey, brown, gold and/or coppery colour or tint.

Advantageously, the nacres in accordance with the invention are micas coated with titanium dioxide or with iron oxide, and also bismuth oxychloride.

According to a particular embodiment, a composition according to the invention comprises less than 1% by weight of nacres.

Particles with a Metallic Tint

For the purposes of the present invention, the term “particles with a metallic tint” means any compound whose nature, size, structure and surface finish allow it to reflect the incident light, notably in a non-iridescent manner.

The particles with a metallic tint that may be used in the invention are chosen in particular from particles of at least one metal and/or of at least one metal derivative, particles including a single-material or multi-material organic or mineral substrate, at least partially covered with at least one coat having a metallic tint comprising at least one metal and/or at least one metal derivative, and mixtures of said particles. Among the metals that may be present in said particles, mention may be made, for example, of Ag, Au, Cu, Al, Ni, Sn, Mg, Cr, Mo, Ti, Zr, Pt, Va, Rb, W, Zn, Ge, Te and Se, and mixtures or alloys thereof. Ag, Au, Cu, Al, Zn, Ni, Mo, Cr and mixtures or alloys thereof (for example, bronzes and brasses) are preferred metals.

The term “metal derivatives” denotes compounds derived from metals, notably oxides, fluorides, chlorides and sulfides.

Illustrations of these particles that may be mentioned include aluminium particles, such as those sold under the names Starbrite 1200 EAC® by the company Siberline and Metalure® by the company Eckart and glass particles coated with a metallic layer, notably those described in JP-A-09188830, JP-A-10158450, JP-A-10158541, JP-A-07258460 and JP-A-05017710.

According to a particular embodiment, a composition according to the invention comprises less than 1% of particles with a metallic tint.

### Hydrophobic Treatment of the Dyestuffs

The pulverulent dyestuffs as described previously may be totally or partially surface treated, with a hydrophobic agent, to make them more compatible with the oily phase of the composition of the invention, notably so that they have good wettability with oils. Thus, these treated pigments are well dispersed in the oily phase. Hydrophobically treated pigments are notably described in EP-A-1 086 683.

The hydrophobic-treatment agent may be chosen from silicones such as methicones, dimethicones and perfluoroalkylsilanes; fatty acids, such as stearic acid; metal soaps, such as aluminium dimyristate, the aluminium salt of hydrogenated tallow glutamate; perfluoroalkyl phosphates; polyhexafluoropropylene oxides; perfluoropolyethers; amino acids; N-acylamino acids or salts thereof; lecithin, isopropyl triisostearyl titanate, isostearyl sebacate, and mixtures thereof.

The term “alkyl” mentioned in the compounds cited previously notably denotes an alkyl group containing from 1 to 30 carbon atoms and preferably containing from 5 to 16 carbon atoms.

### Additives

The composition in accordance with the present invention may also comprise conventional cosmetic adjuvants notably chosen from active agents, softeners, humectants, opacifiers, emollients, silicones, antifoams, fragrances, polar additives, preserving agents, in particular phenoxyethanol, polymers, for example film-forming polymers, propellants, dispersants, anti-pollution agents, chelating agents, basifying or acidifying agents or any other ingredient commonly used in the cosmetic and/or dermatological field

Among the acidifying agents, examples that may be mentioned include mineral or organic acids, for instance hydrochloric acid, orthophosphoric acid, sulfuric acid, carboxylic acids, for instance acetic acid, tartaric acid, citric acid or lactic acid, and sulfonic acids.

Among the basifying agents, examples that may be mentioned include aqueous ammonia, alkali metal carbonates, alkanolamines, such as mono-, di- and triethanolamines and derivatives thereof, sodium hydroxide or potassium hydroxide.

Preferably, the cosmetic composition comprises one or more basifying agents chosen from alkanolamines, in particular triethanolamine, and sodium hydroxide.

In the case of a direct emulsion, the pH of the composition in accordance with the invention is generally between 3 and 12 approximately, preferably between 5 and 11 approximately and even more particularly from 5.5 to 8.

Among the active agents for caring for keratin materials such as the skin, the lips, the scalp, the hair, the eyelashes or the nails, mention may be made, for example, of vitamins and derivatives or precursors thereof, alone or as mixtures; antioxidants; free-radical scavengers; anti-pollutants; self-tanning agents; anti-glycation agents; calmatives; deodorant agents; essential oils; NO-synthase inhibitors; agents for stimulating the synthesis of dermal or epidermal macromolecules and/or for preventing degradation thereof; agents for stimulating fibroblast proliferation; agents for stimulating keratinocyte proliferation; muscle relaxants; refreshing agents; tensioning agents; mattifying agents; depigmenting agents; propigmenting agents; keratolytic agents; desquamating agents; moisturizers; anti-inflammatories; antimicrobials; slimming agents; agents which act on cell energy metabolism; insect repellents; substance P antagonists or CRGP antagonists; agents for preventing hair loss; antiwrinkle agents; anti-ageing agents.

A person skilled in the art will select said active agent(s) as a function of the effect desired on the skin, the hair, the eyelashes, the eyebrows and the nails.

Needless to say, a person skilled in the art will take care to select this or these optional additional compound(s), and/or the amount thereof, such that the advantageous properties of a composition according to the invention are not, or are not substantially, adversely affected by the envisioned addition.

### Composition

The compositions in accordance with the invention may be aqueous or anhydrous. When the compositions are aqueous, they contain at least one aqueous phase.

They may then be in purely aqueous form, i.e. they comprise an amount of fatty phase of less than 10% by weight, preferably less than 5% by weight and even more preferentially less than 2% by weight, relative to the total weight of the composition.

Advantageously, the composition in accordance with the invention is essentially aqueous, i.e. it does not contain any fatty phase.

The compositions according to the invention may also be in particular in the form of a simple or complex (O/W, W/O, O/W/O or W/O/W) emulsion, such as a cream, a milk or a gel-cream.

A composition according to the invention may be in the form of an emulsion, for example an oil-in-water (O/W) or water-in-oil (W/O) emulsion, of a gel, for example an oil-in-water or water-in-oil emulsified gel, of an aqueous composition, or else in the form of a composition of gel/gel type.

According to one embodiment, the composition according to the present invention is an emulsion.

According to one variant, the composition according to the invention is a simple emulsion.

According to one variant, the composition according to the invention is a multiple emulsion. It may be a triple emulsion.

According to one variant, the composition according to the invention is a direct simple emulsion. Thus, according to one embodiment, the composition according to the present invention is an oil-in-water (O/W) emulsion.

According to one variant, the composition according to the invention is an inverse simple emulsion. Thus, according to one embodiment, the composition according to the present invention is a water-in-oil (W/O) emulsion.

According to one variant, the composition according to the invention is an aqueous formulation of aqueous gel or solution type.

Preferably, the composition is an emulsion, notably a water-in-oil or oil-in-water emulsion.

In the case of compositions in the form of oil-in-water or water-in-oil emulsions, the emulsification processes that may be used are of the paddle or impeller, rotor-stator and HPH type.

In order to obtain stable emulsions with a low content of polymer (oil/polymer ratio >25), it is possible to prepare the dispersion in concentrated phase and then to dilute the dispersion with the remainder of the aqueous phase.

It is also possible, via HPH (between 50 and 800 bar), to obtain stable dispersions with drop sizes that may be as small as 100 nm.

The composition according to the invention may be more or less fluid and may have the appearance of a white or coloured cream, an ointment, a milk, a lotion, a serum, a paste or a foam. It may optionally be applied to the skin in aerosol form. It may also be in solid form, for example in the form of a stick.

A composition according to the invention may be in the form of a cosmetic composition for caring for and/or making up keratin materials, preferably a cosmetic composition for caring for keratin materials, in particular of the body or of the face, preferably of the face.

The compositions according to the invention find their application in a large number of treatments, notably cosmetic treatments, for the skin, the lips and the hair, including the scalp, notably for protecting and/or caring for the skin, the lips and/or the hair, and/or for making up the skin and/or the lips.

Another subject of the present invention consists of the use of the compositions according to the invention as defined above for the manufacture of products for the cosmetic treatment of the skin, the lips, the nails, the hair, the eyelashes, the eyebrows and/or the scalp, notably care products, antisun products and makeup products.

The compositions according to the invention may be in the form of products for caring for the skin or semi-mucous membranes, such as a protective or cosmetic care composition for the face, for the lips, for the hands, for the feet, for the anatomical folds or for the body (for example, day creams, night cream, day serum, night serum, makeup-removing cream, makeup base, antisun composition, protective or care body milk, aftersun milk, skincare or scalp-care lotion, gel or foam, serum, mask, or aftershave composition).

The cosmetic compositions according to the invention may be used, for example, as care products and/or antisun protection products for the face and/or the body, of liquid to semi-liquid consistency, such as lotions, milks, more or less rich creams, gels and cream-gels. They may optionally be packaged in aerosol form and may be in the form of a mousse or a spray.

The compositions according to the invention in the form of vaporizable fluid lotions in accordance with the invention are applied to the skin or hair in the form of fine particles by means of pressurizing devices. The devices in accordance with the invention are well known to those skilled in the art and comprise non-aerosol pumps or “atomizers”, aerosol containers comprising a propellant and aerosol pumps using compressed air as propellant. These devices are described in patents U.S. Pat. Nos. 4,077,441 and 4,850,517.

The compositions packaged in aerosol form in accordance with the invention generally contain conventional propellants, for instance hydrofluoro compounds, dichlorodifluoromethane, difluoroethane, dimethyl ether, isobutane, n-butane, propane or trichlorofluoromethane. They are preferably present in amounts ranging from 15% to 50% by weight relative to the total weight of the composition.

The composition may be applied to the skin by hand or using an applicator.

According to another aspect, the invention also relates to a cosmetic assembly comprising:
i) a container delimiting one or more compartment(s), said container being closed by a closing member and optionally being unsealed, and
ii) a makeup and/or care composition in accordance with the invention placed inside said compartment(s).

The container may be, for example, in the form of a jar or a box.

The closing member may be in the form of a lid comprising a cap mounted so as to be able to move by translation or by pivoting relative to the container housing said makeup and/or care composition(s).

Such compositions are notably prepared according to the general knowledge of a person skilled in the art.

Such compositions as defined previously may notably be employed for a cosmetic use according to the invention.

Thus, the invention also relates to the use of a composition according to the invention for caring for and/or making up keratin materials, preferably for caring for keratin materials, in particular the skin of the body and/or of the face.

The invention also relates to a cosmetic process for making up and/or caring for keratin materials, in particular the skin, comprising at least one step of applying a composition as defined previously to said keratin materials.

Preferably, the invention also relates to a cosmetic process for caring for keratin materials, in particular of the body and/or of the face, comprising at least one step of applying a composition as defined above to said keratin materials.

The cosmetic processes for making up and/or caring for keratin materials, in particular the skin, are non-therapeutic.

In particular, the present invention relates to the non-therapeutic cosmetic use of a composition as described above, for protecting keratin materials, in particular the skin and/or the lips and/or the hair against solar radiation.

The invention also relates to the non-therapeutic cosmetic use of a composition as defined above, for combating or preventing the signs of photo-induced premature ageing of keratin materials, in particular the skin and/or the lips and/or the hair.

In addition, the present invention relates to the cosmetic use of a composition as defined above, for preventing premature ageing of keratin materials, in particular the skin, notably on the face, the neckline, the arms, the hands and/or the shoulders, in particular the signs of skin ageing of actinic origin, such as photo-ageing.

The present invention also relates to the cosmetic use of a composition as defined above to prevent a loss of firmness and/or elasticity and/or tonicity and/or suppleness of the skin, the formation of wrinkles and fine lines, a dull complexion, and/or a wizened appearance of the skin.

The present invention also relates to a non-therapeutic cosmetic process for protecting keratin materials, in particular the skin and/or the lips and/or the hair against solar radiation, comprising the application, to the surface of the keratin material, of at least one composition according to the invention.

The invention also relates to a non-therapeutic cosmetic process for preventing and/or treating the signs of ageing of a keratin material, comprising the application, to the surface of the keratin material, of at least one composition according to the invention.

For the purposes of the present invention, the term “preventing” means, at least partly, reducing the risk of occurrence of a given phenomenon.

Throughout the description, including the claims, the expression “including a” should be understood as being synonymous with “including at least one”, unless otherwise specified.

The expressions “between . . . and . . . ”, “comprises from . . . to . . . ”, “formed from . . . to . . . ” and “ranging from . . . to . . . ” should be understood as being inclusive of the limits, unless otherwise specified.

In the description and the examples, unless otherwise indicated, the percentages are percentages by weight. The percentages are thus expressed by weight relative to the total weight of the composition. The temperature is expressed in degrees Celsius, unless otherwise indicated, and the pressure is atmospheric pressure, unless otherwise indicated.

The invention is illustrated in greater detail by the non-limiting examples presented below.

### EXAMPLE

### Example A: Preparation of Merocyanines According to the Invention

### Example A1: Preparation of Compound (A)

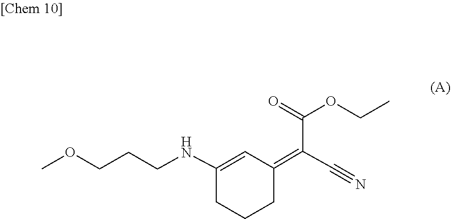 [SMILES: CCOC(=O)/C(C#N)=C1\C=C(NCCCOC)CCC1]

122.23 grams of 3-[(3-methoxypropyl)amino]-2-cyclohexen-1-one were alkylated with dimethyl sulfate or alternatively with diethyl sulfate and treated with 75.45 grams of ethyl cyanoacetate in approximately equimolar proportions in the presence of a base and optionally of a solvent.

The following base/solvent combinations were used:

TABLE 2

| Example | Base | Solvent |
|---|---|---|
| Example A1.1 | DBU (1,8- | dimethylacetamide |
| | diazabicyclo[5.4.0]undec-7-ene) | |
| Example A1.2 | triethylamine | isopropanol |
| Example A1.3 | 3-methoxypropylamine | isopropanol |
| Example A1.4 | 3-methoxypropylamine | tert-amyl alcohol |
| Example A1.5 | 3-methoxypropylamine | toluene |
| Example A1.6 | 3-methoxypropylamine | dimethylformamide |
| Example A1.7 | 3-methoxypropylamine | no solvent |
| Example A1.8 | N-morpholine | isopropanol |

The completion of the alkylation reaction could be monitored, for example, via methods such as TLC, GC or HPLC.162.30 g of compound (14) were obtained in the form of a brown oil. After crystallization, the product was obtained in the form of yellowish crystals.

Melting point: 92.7° C.

### Example A2: Preparation of Compound (B)

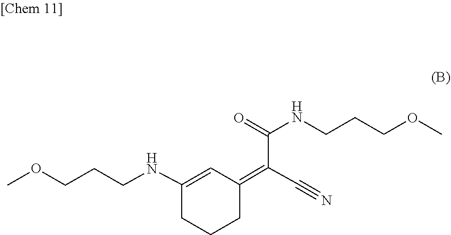

101.00 g of 3-[(3-methoxypropyl)amino]-2-cyclohexen-1-one were alkylated with dimethyl sulfate or alternatively with diethyl sulfate and treated with 86.00 g of 2-cyano-N-(3-methoxypropyl)acetamide in approximately equimolar proportions in the presence of a base and optionally of a solvent.

The following base/solvent combinations were used:

TABLE 3

| Example | Base | Solvent |
|---|---|---|
| Example A2.1 | DBU (1,8- | dimethylacetamide |
| | diazabicyclo[5.4.0]undec-7-ene) | |
| Example A2.2 | triethylamine | isopropanol |
| Example A2.3 | 3-methoxypropylamine | isopropanol |
| Example A2.4 | 3-methoxypropylamine | tert-amyl alcohol |
| Example A2.5 | 3-methoxypropylamine | toluene |
| Example A2.6 | 3-methoxypropylamine | dimethylformamide |
| Example A2.7 | 3-methoxypropylamine | no solvent |

The crude product (B) was obtained in the form of a dark brown oil. After silica gel column chromatography (eluent: 99/1 toluene/methanol), 81.8 g of product were obtained in the form of yellowish crystals.

Melting point: 84.7-85.3° C.

### Example A3: Preparation of Compound (D)

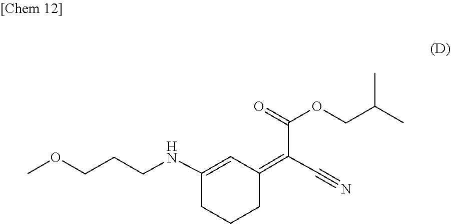 [SMILES: COCCCNC1=C/C(=C(/C#N)C(=O)OCC(C)C)CCC1]

13.09 g of 3-[(3-methoxypropyl)amino]-2-cyclohexen-1-one were alkylated with dimethyl sulfate or alternatively with diethyl sulfate and treated with 10.12 g of isobutyl cyanoacetate in the presence of a base and optionally of a solvent.

The following base/solvent combinations were used:

TABLE 4

| Example | Base | Solvent |
|---|---|---|
| Example A3.1 | DBU (1,8- | dimethylacetamide |
| | diazabicyclo[5.4.0]undec-7-ene) | |
| Example A3.2 | triethylamine | isopropanol |
| Example A3.3 | 3-methoxypropylamine | isopropanol |
| Example A3.4 | N-methylmorpholine | tert-amyl alcohol |
| Example A3.5 | 3-methoxypropylamine | toluene |
| Example A3.6 | 3-methoxypropylamine | dimethylformamide |
| Example A3.7 | 3-methoxypropylamine | no solvent |

15.97 g of crude product (27) were obtained in the form of a dark brown oil. After silica gel column chromatography (eluent: toluene/acetone), 13.46 g of product were obtained in the form of yellowish crystals.

Melting point: 96.3° C.

### Example A4: Preparation of Compound (C)

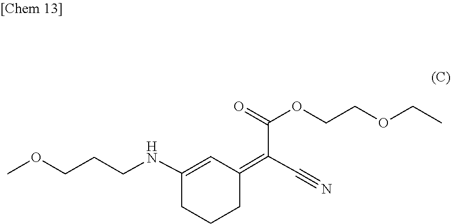 [SMILES: CCOCCOC(=O)/C(C#N)=C1\C=C(NCCCOC)CCC1]

148.4 g of 3-[(3-methoxypropyl)amino]-2-cyclohexen-1-one were alkylated with dimethyl sulfate or alternatively with diethyl sulfate and treated with 130.00 g of 2-ethoxyethyl cyanoacetate in the presence of an organic base and of a solvent.

The following base/solvent combinations were used:

TABLE 5

| Example | Base | Solvent |
|---|---|---|
| Example A4.1 | DBU (1,8- | dimethylacetamide |
| | diazabicyclo[5.4.0]undec-7-ene) | |
| Example A4.2 | triethylamine | isopropanol |
| Example A4.3 | 3-methoxypropylamine | isopropanol |
| Example A4.4 | N-methylmorpholine | tert-amyl alcohol |
| Example A4.5 | 3-methoxypropylamine | toluene |
| Example A4.6 | 3-methoxypropylamine | dimethylformamide |
| Example A4.7 | 3-methoxypropylamine | no solvent |

### Example: Composition 1 According to the Invention: Anhydrous Serum

The following composition was prepared according to the process below:
1) Mixing and heating the glycol-based phase A to 85-90° C. with magnetic stirring.
2) Adding B with magnetic stirring (vitamin C) until completely dissolved
3) Switching off the heating and adding C (the methoxypropylamino cyclohexenylidene ethoxyethylcyanoacetate screening agent according to Example A), with magnetic stirring, until completely dissolved.

The values are expressed as weight percentages relative to the total weight of the composition.

TABLE 6

| | | Composition 1 |
|---|---|---|
| | | according to |
| Phase | Compounds (INCI) | the invention |
| A | Polysorbate 20 (Tego SML 20 MB from Evonik) | 0.50 |
| A | Dipropylene glycol (Dow) | 24.50 |
| A | Propylene carbonate (Activemol PC from | 6.00 |
| | Innospec Active Chemicals) | |
| A | Glycerol (Refined Glycerine 99.5% Ph. Eur. | 24.37 |
| | from Cargill) | |
| A | Propylene glycol (Dongying Hi-Tech Spring | 30.00 |
| | Chemical IN) | |
| B | Ascorbic acid (CSPC Weisheng | 12.63 |
| | Pharmaceutical) | |
| C | Methoxypropylamino cyclohexenylidene | 2.00 |
| | ethoxyethylcyanoacetate (according to | |
| | Example A) | |

### Example: Composition 2 According to the Invention: Aqueous Serum

The following composition was prepared according to the process below:

Preparation of phase A:
1) Mix the ingredients of A1 with magnetic stirring (add the sodium hydroxide slowly).
2) Add the PQ-67 (A2) using a deflocculator until a transparent, viscous gel is obtained.

### Preparation of Phase B:

1) Mix all the ingredients of B1 at elevated temperature (50-60° C.) with magnetic stirring.
2) Add the niacinamide (B2) with magnetic stirring until totally dissolved.
3) Add the caffeine (B3) with magnetic stirring until totally dissolved. Adjust water content (QS)

Preparation of phase C: Prepare the methoxypropylamino cyclohexenylidene ethoxyethylcyanoacetate screening agent+dipropylene glycol mixture with magnetic stirring and heat to accelerate the dissolution of the methoxypropylamino cyclohexenylidene ethoxyethylcyanoacetate screening agent (60-70° C.).

Allow the mixture to return to RT and then add the alcohol (D)

Add phase B to phase A using a deflocculator and finally add phase C to the final gel.

The values are expressed as weight percentages relative to the total weight of the composition.

TABLE 7

| | | Composition 2 |
|---|---|---|
| | | according to |
| Phase | Compounds (INCI) | the invention |
| A1 | Sodium hydroxide (Sodium Hydroxide | 3.3 |
| | 48% from PT. Mulia Agung Chemindo) | |
| A1 | Ascorbic acid (CSPC Weisheng | 15.0 |
| | Pharmaceutical) | |
| A1 | Water | 20 |
| A2 | Polyquaternium-67 (Softcat Polymer SL- | 0.5 |
| | 100 from Amerchol (Dow Chemical)) | |
| B1 | Hexylene glycol (Rhodia (Solvay)) | 5.0 |
| B1 | Water | 11.2 |
| B1 | Trisodium ethylenediamine disuccinate | 0.2 |
| | (Natrlquest E30 from Innospec Active | |
| | Chemicals) | |
| B1 | Hydroxyacetophenone (Symsave H O from | 0.5 |
| | Symrise) | |
| B1 | Pentylene glycol (A-Leen 5 from | 3.0 |
| | Minasolve) | |
| B1 | Glycerol (Refined Glycerine 99.5% Ph. | 7.0 |
| | Eur. from Cargill) | |
| B1 | Caprylyl glycol (Cleanbio-CG from Kolon | 0.3 |
| | Life Science) | |
| B2 | Niacinamide (DSM Nutritional Products) | 4.0 |
| B3 | Caffeine (BASF) | 2.0 |
| C | Dipropylene glycol (Dow) | 24.0 |
| C | Methoxypropylamino cyclohexenylidene | 1.5 |
| | ethoxyethylcyanoacetate (according to | |
| | Example A) | |
| D | Denat. alcohol (Cargill 65211 from | 2.5 |
| | Cargill) | |

### Example: Composition 3 According to the Invention: Emulsion

The following solutions were prepared according to the process below.
phase A: in a beaker, mix the ingredients with magnetic stirring at 40° C.
Phase B: in a beaker, mix the ingredients with magnetic stirring at 55° C.

Add B to A with a rotor-stator emulsifier at 3000 rpm+scraping blades at 90 rpm for 10 minutes.

Add phase C to A+B with a rotor-stator emulsifier at 3000 rpm+scraping blades at 90 rpm for 10 minutes at room temperature.

Next, rapidly add D and then E: emulsifier at 1500 rpm+scraping blades at 50 rpm for 10 minutes at room temperature.

Finally, add F: emulsifier at 1500 rpm+scraping blades at 50 rpm for 10 minutes at room temperature.

The values are expressed as weight percentages relative to the total weight of the

TABLE 8

| | | Composition 3 |
|---|---|---|
| | | according to the |
| Phases | Compounds (INCI) | invention |
| A | Water | 35 |
| A | Pentylene glycol | 3.00 |
| A | Niacinamide | 4.00 |
| A | Dipropylene glycol | 8.00 |
| A | Trisodium | 0.25 |
| | ethylenediaminedisuccinate | |
| A | Hydroxyacetophenone | 0.50 |
| A | Methoxypropylamino | 1.00 |
| | cyclohexenylidene | |
| | ethoxyethylcyanoacetate (according | |
| | to Example A) | |
| A | Caffeine | 4.00 |
| B | PEG-40 stearate | 1.57 |
| B | Sucrose tristearate | 1.08 |
| B | Sodium dilauramidoglutamide lysine | 0.35 |
| B | C15-19 alkane | 7.90 |
| B | Glyceryl stearate | 2.00 |
| C | Sodium hyaluronate | 0.50 |
| C | Ammonium | 1.50 |
| | polyacryloyldimethyltaurate | |
| C | Sodium polyacrylate | 0.50 |
| D | Isododecane | 1.70 |
| E | Sodium hydroxide | 2.69 |
| E | Ascorbic acid | 12.00 |
| E | Water | 10 |
| F | Boron nitride | 0.50 |
| F | Lauroyl lysine | 2.00 |

### Example: Evaluation of Procollagen I Production

The efficacy of a combination in accordance with the invention and outside the invention is tested by assaying procollagen 1 in a culture medium of normal human dermal fibroblasts exposed to UVA.

The other materials and the protocol used are specified below.

### Experimental Protocol

### Cell Culture

Normal human dermal fibroblasts (NHDF) obtained from a mammary plasty.
Culture: 37° C., 5% CO₂.
Culture medium and test medium: MEM (Gibco 21090-022) containing 10% foetal calf serum (FCS) (Gibco 10270-098), 2 mM L-glutamine (Gibco 25030-024), 1 mM sodium pyruvate (Gibco 11360-039), 1× non-essential amino acids (Gibco 11140-035), 250 ng/ml amphotericin B (Gibco 15290-018) and penicillin/streptomycin 20 U/20 pg/ml.

The normal human dermal fibroblasts were seeded in complete MEM medium and incubated for 72 h at 37° C. and 5% CO₂.

### Plating the Formulas

The following formulae (1.3 mg/cm²) were homogeneously spread on PMMA plates: various similar formulae were prepared, the difference being whether or not the test compound(s) were included, notably: Control, or by incorporating therein according to the invention methoxypropylamino cyclohexenylidene ethoxyethylcyanoacetate (according to Example A) at 1%, 2% or 3% by weight relative to the total weight of the composition.

Such a formula with 2% by weight of methoxypropylamino cyclohexenylidene ethoxyethylcyanoacetate (according to Example A) is described below.

A composition comprising a UV-screening agent system outside the invention is also prepared and tested.

Formula with 2% by Weight of Methoxypropylamino Cyclohexenylidene Ethoxyethylcyanoacetate

### Process for Preparing the Formula Below:

1/ Heat phase A and phase B to 70-80° C. with magnetic stirring
2/ Introduce B into A using a rotor-stator (1500 rpm) for 10-15 minutes
3/ Introduce phase C using a rotor-stator (1500 rpm)
4/ Cool the emulsion (ice water bath) to about 35-40° C.
5/ Then introduce phase D using a deflocculating paddle
6/ Add phases E and F using a deflocculating paddle.

The values are expressed as weight percentages relative to the total weight of the

TABLE 9

| | | Formula with 2% of |
|---|---|---|
| | | methoxypropylamino |
| | | cyclohexenylidene |
| Phase | Ingredient | ethoxyethylcyanoacetate |
| A | Glyceryl stearate (and) | 1.5 |
| | PEG-100 stearate | |
| | (Arlacel 165-FP-PA-(SG) | |
| | from Croda) | |
| A | Stearic acid | 1.5 |
| A | Dimethicone (Dowsil SH | 0.5 |
| | 200 C Fluid 350 cSt from | |
| | Dow Corning) | |
| A | Methylparaben | 0.24 |
| A | Ethylparaben | 0.34 |
| A | Phenethyl benzoate | 15 |
| | (and) benzoic acid (X- | |
| | Tend 226 from ISP | |
| | (Ashland)) | |
| A | Methoxypropylamino | 2 |
| | cyclohexenylidene | |
| | ethoxyethylcyanoacetate | |
| | (according to Example | |
| | A) | |
| B | Triethanolamine | 0.45 |
| B | Glycerol | 6 |
| B | Disodium EDTA | 0.1 |
| | (Shijiazhuang | |
| | Jackchem) | |
| B | Water | 63.63 |
| B | Potassium cetyl | 1 |
| | phosphate (Amphisol K | |
| | from DSM Nutritional | |
| | Products) | |
| B | Phenoxyethanol ((Toho | 0.7 |
| | Chemicals) | |
| C | Acrylates/C10-30 alkyl | 0.25 |
| | acrylate crosspolymer | |
| | (Pemulen TR-1 Polymer | |
| | from Lubrizol) | |
| C | Xanthan gum | 0.1 |
| C | Isohexadecane (Ineos) | 2 |
| D | Triethanolamine | 0.25 |
| D | Water | 1.44 |
| E | PEG-12 dimethicone | 1 |
| | (Silsoft 880 from | |
| | Momentive Performance | |
| | Materials) | |
| F | Denat. alcohol | 2 |

### Formula Comprising a UV Screening Agent System Outside the Invention, Notably at a Content of 11.8% by Weight Relative to the Total Weight of the Composition.

### Preparation Process

Phase A: Take a very tall beaker to introduce the aqueous phase A1 (boiling water). Add sunspheres (A2) using a rotor-stator for 20 min at 3000 rpm. Adjust the qs of water and maintain a temperature of 70° C.
Phase B: Mix the ingredients of phase B with magnetic stirring and heating (70-80° C.)
Introduce B onto A using a rotor-stator (1500 rpm) for 10-15 minutes
Cool by introducing phase D (cold water) using a rotor-stator
Add phase C (gelling agents) using a deflocculating paddle
Successively add phases E to H using a deflocculating paddle, at a temperature below 30° C.

The values are expressed as weight percentages relative to the total weight of the composition.

TABLE 10

| | | Formula |
|---|---|---|
| | | comprising |
| | | a UV- |
| | | screening |
| | | agent |
| | | system |
| | | outside the |
| INCI | Phase | invention |
| Water/Aqua | A1 | 35.02 |
| Glycerol | | 5 |
| Disodium EDTA | | 0.2 |
| Triethanolamine | | 0.18 |
| Styrene/acrylates copolymer (Sunspheres | A2 | 2 |
| Powder from Rohm & Haas (Dow Chemical) | | |
| Stearic acid | B | 1 |
| Glyceryl stearate (and) PEG-100 stearate | | 3 |
| Cetyl alcohol | | 1 |
| Synthetic wax | | 1 |
| Caprylyl glycol | | 0.3 |
| Phenoxyethanol | | 0.3 |
| Dicaprylyl carbonate | | 5 |
| Isopropyl lauroyl sarcosinate | | 9.3 |
| Octocrylene | | 3 |
| Butylmethoxydibenzoylmethane | | 3.5 |
| Diethylamino hydroxybenzoyl hexyl benzoate | | 3 |
| Bis(ethylhexyloxyphenol)methoxyphenyltriazine | | 2 |
| Ethylhexyl triazone | | 0.3 |
| Dimethicone | C | 2 |
| Ammonium polyacryloyldimethyltaurate | | 0.4 |
| Xanthan gum | | 0.2 |
| Water/Aqua | D | 15 |
| Dimethicone (and) dimethicone/vinyl | E | 1 |
| dimethicone crosspolymer | | |
| Silica | F | 3 |
| Alcohol | G | 3 |
| Fragrance/perfume | H | 0.3 |

### Continuation of the Experimental Protocol

The cells are then exposed for 3 consecutive days to a dose of 25 J/cm² of total UVA using an Oriel solar simulator fitted with a WG335 filter. Depending on the conditions, a PMMA plate to which the formula comprising the screening agents according to the invention or outside the invention or the Placebo formula is applied is placed over the cells during exposure. After each exposure, the cells are treated or not with ascorbic acid (10 μM) diluted in the culture medium. The cells are then incubated at 37° C., 5% CO2. 48 hours after the last exposure, a cell viability test (XTT Roche cat. 11465015001) is performed, and the number of cells in each well is evaluated by means of a Cyquant assay (Molecular Probes C7026). The media are collected and a procollagen I assay is performed by ELISA (Abcam ab210966). Each experimental condition is performed in duplicate or triplicate and at least three independent experiments are performed.

The results are given in the following table.

TABLE 11

| | | Mean | | | | |
|---|---|---|---|---|---|---|
| | | procollagen | | | | |
| | | 1 | | | | |
| | | (normalized | | | | |
| | | by Cyquant | | relative | | |
| | | signal and | | % | | |
| | | relative to | | relative | | |
| | | the | | to the | | |
| | | unexposed | | control | | |
| VA | | Control) | SD | 0 J | n | p-value |
| 0 J/cm² | Control | 100 | 8.3 | | 13 | |
| | VitC (Vitamin C) | 360.5 | 52.4 | +260.5% | 10 | <0.05 vs |
| | 10 μM | | | | | Control |
| 25 J/cm² | Control | 46.1 | 10.2 | −53.9% | 11 | |
| | VitC 10 μM | 129.7 | 32.4 | +29.7% | 8 | <0.05 vs VitC |
| | | | | | | 0 J/cm² |
| | Placebo | 52.9 | 14.3 | −47.1% | 13 | |
| | Formula with | 71.3 | 20 | −28.7% | 11 | |
| | methoxypropylamino | | | | | |
| | cyclohexenylidene | | | | | |
| | ethoxyethylcyano- | | | | | |
| | acetate (according | | | | | |
| | to Example A) | | | | | |
| | at 1% | | | | | |
| | Formula with | 77.6 | 14.2 | −22.4% | 6 | |
| | methoxypropylamino | | | | | |
| | cyclohexenylidene | | | | | |
| | ethoxyethylcyano- | | | | | |
| | acetate at 2% | | | | | |
| | Formula with | 81.7 | 11.8 | −18.3% | 11 | |
| | methoxypropylamino | | | | | |
| | cyclohexenylidene | | | | | |
| | ethoxyethylcyano- | | | | | |
| | acetate at 3% | | | | | |
| | Formula with UV- | 82.5 | 10.7 | −17.5% | 11 | |
| | screening agent | | | | | |
| | system outside the | | | | | |
| | invention | | | | | |
| | Vitamin C + Placebo | 124.3 | 33.7 | +24.3% | 10 | <0.05 vs VitC |
| | | | | | | 0 J/cm² |
| | Vitamin C + | 192.7 | 33.1 | +92.7% | 8 | <0.05 vs |
| | methoxypropylamino | | | | | methoxy- |
| | cyclohexenylidene | | | | | propylamino- |
| | ethoxyethylcyano- | | | | | cyclohexenylidene |
| | acetate at 1% | | | | | ethoxyethyl- |
| | | | | | | cyanoacetate |
| | | | | | | at 1% <0.05 vs |
| | | | | | | VitC + Placebo |
| | Vitamin C + | 248 | 38.3 | +148% | 6 | <0.05 vs |
| | methoxypropylamino | | | | | methoxy- |
| | cyclohexenylidene | | | | | propylamino- |
| | ethoxyethylcyano- | | | | | cyclohexenylidene |
| | acetate at 2% | | | | | ethoxyethyl- |
| | | | | | | cyanoacetate |
| | | | | | | (according |
| | | | | | | to Example) |
| | | | | | | at 2% <0.05 vs |
| | | | | | | VitC + Placebo |
| | Vitamin C + | 249.6 | 31.3 | +149.6% | 8 | <0.05 vs |
| | methoxypropylamino | | | | | methoxy- |
| | cyclohexenylidene | | | | | propylamino- |
| | ethoxyethylcyano- | | | | | cyclohexenylidene |
| | acetate (according | | | | | ethoxyethyl- |
| | to Example A) at 3% | | | | | cyanoacetate |
| | | | | | | at 3% <0.05 vs |
| | | | | | | VitC + Placebo |
| | Vitamin C + | 238 | 34.5 | +138% | 8 | <0.05 vs |
| | Formula with UV- | | | | | UV-screening |
| | screening agent | | | | | agent system |
| | system outside the | | | | | outside the |
| | invention | | | | | invention |
| | | | | | | <0.05 vs |
| | | | | | | VitC + Placebo |

Under these experimental conditions, the amount of procollagen 1 synthesized and secreted by the fibroblasts 48 hours after the last exposure is correctly detectable.

Ascorbic acid tested at 10 μM significantly increased the secretion of procollagen 1 by the fibroblasts.

UVA significantly reduced the ability of ascorbic acid to induce procollagen 1 secretion.

Surprisingly, the combination according to the invention of ascorbic acid with the methoxypropylamino cyclohexenylidene ethoxyethylcyanoacetate screening agent, in particular with a content of 2% or 3% by weight, increased the secretion of procollagen 1 in a similar manner to a combination outside the invention comprising ascorbic acid with a UV-screening agent system at a total content of 11.8% by weight, while at the same time not requiring the presence of a high content of UV-screening agents.

1. A cosmetic and/or dermatological composition, in particular for making up and/or caring for keratin materials, comprising:
at least one merocyanine of formula (I) below and also the E/E- or E/Z-geometrical isomer forms thereof: 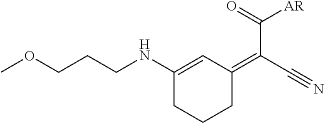
in which:
A is —O— or —NH;
R is a C₁-C₂₂ alkyl group, a C₂-C₂₂ alkenyl group, a C₂-C₂₂ alkynyl group, a C₃-C₂₂ cycloalkyl group or a C₃-C₂₂ cycloalkenyl group, said groups possibly being interrupted with one or more O; and
at least ascorbic acid and/or a derivative thereof.

2. The cosmetic and/or dermatological composition according to claim 1, comprising at least as merocyanine of formula (I), at least one compound chosen from the following compounds and also the E/E- or E/Z-geometrical isomer forms thereof:

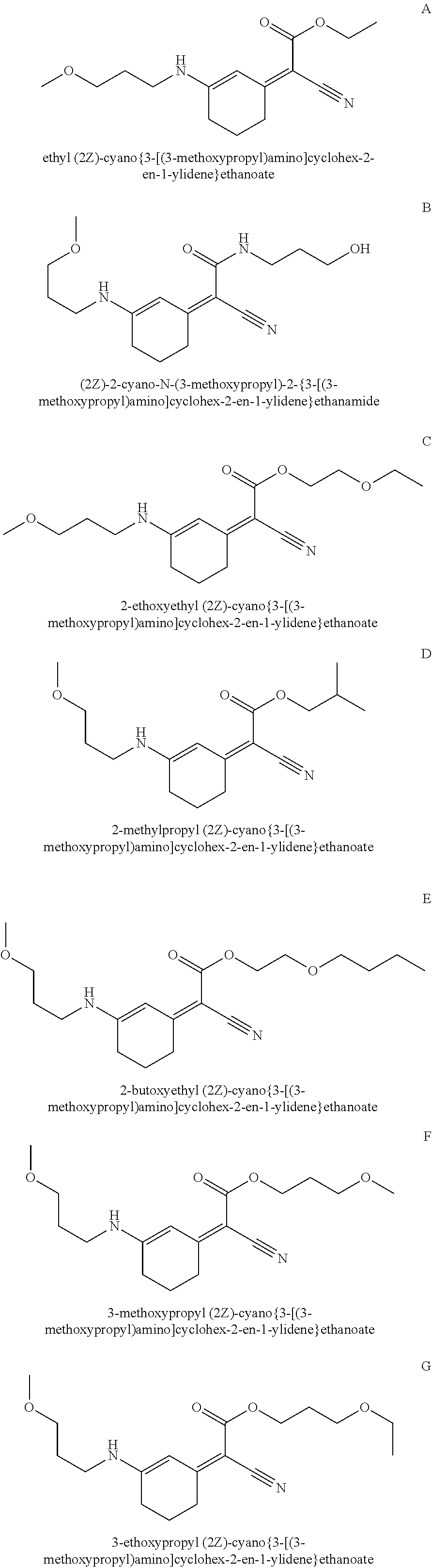

3. The cosmetic and/or dermatological composition according to claim 1, in which the merocyanine of formula (I) is the compound 2-ethoxyethyl (2Z)-cyano{3-[(3-methoxypropyl)amino]cyclohex-2-en-1-ylidene}ethanoate (C) in its E/Z geometrical configuration having the following structure: 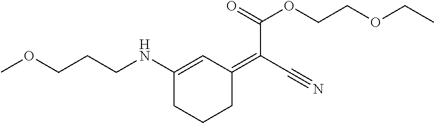 [SMILES: CCOCCOC(=O)/C(C#N)=C1\C=C(NCCCOC)CCC1]
and/or in its E/E geometrical configuration having the following structure:

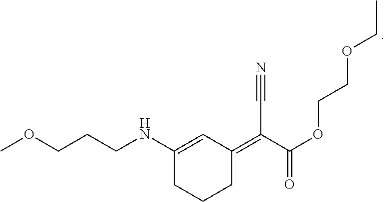 [SMILES: CCOCCOC(=O)/C(C#N)=C1/C=C(NCCCOC)CCC1]

4. The cosmetic and/or dermatological composition according to claim 1, in which the content of merocyanine(s) of formula (I) ranges from 0.1% to 10% by weight relative to the total weight of the composition.

5. The cosmetic and/or dermatological composition according to claim 1, in which the ascorbic acid and/or a derivative thereof are chosen from ascorbic acid, ascorbyl-2 glucoside and magnesium ascorbyl phosphate, and mixtures thereof.

6. The cosmetic and/or dermatological composition according to claim 1, comprising ascorbic acid.

7. The cosmetic and/or dermatological composition according to claim 1, in which the content of ascorbic acid and/or a derivative thereof ranges from 0.01% to 30% by weight, relative to the total weight of the composition.

8. The cosmetic and/or dermatological composition according to claim 1, comprising at least one hydrotrope chosen from nicotinamide, caffeine, and mixtures thereof.

9. The cosmetic and/or dermatological composition according to claim 1, comprising at least one hydrotrope chosen from nicotinamide, caffeine, salicylic acid salts, the sodium salt of pyroglutamic acid (sodium PCA), sodium 1,3-benzenedisulfonate, sodium benzoate, sodium 4-pyridinecarboxylate, sodium benzenesulfonate, sodium p-toluenesulfonate (NaPTS), sodium butyl monoglycol sulfate (NaBMGS), 4-aminobenzoic acid HCl, sodium cumene sulfonate, N,N-diethyl nicotinamide, N-picolyl nicotinamide, N-allyl nicotinamide, 2-methacryloyloxyethyl phosphorylcholine, resorcinol, pyrogallol, N-picolylacetamide, procaine HCl, proline HCl, pyridine, 3-picolylamine, ibuprofen sodium, sodium xylene sulfonate (SXS), ethyl carbamate, pyridoxal hydrochloride, sodium benzoate, N,N-dimethylacetamide, N-methylacetamide, isoniazid, and mixtures thereof.

10. The cosmetic and/or dermatological composition according to claim 1, comprising at least one hydrotrope chosen from nicotinamide, caffeine, salicylic acid salts, and mixtures thereof.

11. The cosmetic and/or dermatological composition according to claim 1, in which the content of hydrotrope(s) ranges from 0.1% to 20% by weight relative to the total weight of the composition.

12. The cosmetic and/or dermatological composition according to claim 1, comprising at least one alcohol.

13. The cosmetic and/or dermatological composition according to claim 1, also comprising at least one alkylene carbonate and preferably propylene carbonate.

14. The cosmetic and/or dermatological composition according to claim 1, comprising at least one polyol.

15. The cosmetic and/or dermatological composition according to claim 1, comprising at least one fatty phase, preferably ranging from 5% to 95% by weight relative to the total weight of the composition.

16. The cosmetic and/or dermatological composition according to claim 1, also comprising at least one additional UV-screening agent different from the merocyanines of formula (I).

17. The cosmetic and/or dermatological composition according to claim 1, characterized in that it is a cosmetic composition for caring for keratin materials.

18. A cosmetic process for caring for keratin materials comprising at least one step of applying a composition as defined in claim 1 to said keratin materials.

19. The cosmetic and/or dermatological composition according to claim 2, in which the merocyanine of formula (I) is the compound 2-ethoxyethyl (2Z)-cyano{3-[(3-methoxypropyl)amino]cyclohex-2-en-1-ylidene}ethanoate (C) in its E/Z geometrical configuration having the following structure: 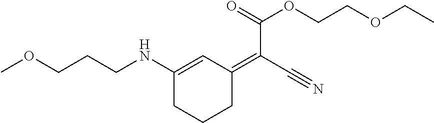 [SMILES: CCOCCOC(=O)/C(C#N)=C1\C=C(NCCCOC)CCC1]
and/or in its E/E geometrical configuration having the following structure:

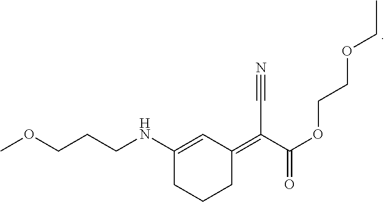 [SMILES: CCOCCOC(=O)/C(C#N)=C1/C=C(NCCCOC)CCC1]

20. The cosmetic and/or dermatological composition according to claim 2, in which the content of merocyanine(s) of formula (I) ranges from 0.1% to 10% by weight relative to the total weight of the composition.
